(19) United States
(12) Patent Application Publication
(10) Pub. No.: US 2025/0353819 A1
(43) Pub. Date: Nov. 20, 2025

(54) ARYL HYDANTOIN COMPOUNDS AND METHODS OF USE

(71) Applicants: BOARD OF REGENTS THE UNIVERSITY OF NEBRASKA, Lincoln, NE (US); MONASH UNIVERSITY, Clayton (AU); Swiss Tropical and Public Health Institute, Allschwil (CH)

(72) Inventors: Jonathan L. Vennerstrom, Omaha, NE (US); Derek A. Leas, Gainesville, FL (US); Susan Charman, South Melbourne (AU); Jennifer Keiser, Basel (CH)

(21) Appl. No.: 18/870,785

(22) PCT Filed: Jun. 1, 2023
(86) PCT No.: PCT/US23/24140

§ 371 Date: Dec. 2, 2024

Related U.S. Application Data
(60) Provisional application No. 63/347,585, filed on Jun. 1, 2022.

(51) Int. Cl. C07D 233/76; A61K 31/4166; A61P 33/12

(52) U.S. Cl. CPC C07D 233/76; A61K 31/4166; A61P 33/12

(57) ABSTRACT

Provided herein are aryl hydantoin compounds having the structure of Formula (I) wherein X, R¹, R², R³, R⁴, and n are described herein. Also provided herein are compositions comprising compounds of Formula (I) and methods of using compounds of Formula (I) in treating, inhibiting, and/or preventing a parasitic disease (e.g., Schistosomiasis).

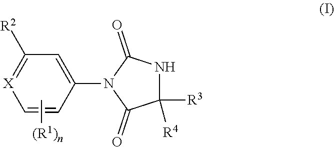

### STATEMENT OF U.S. GOVERNMENT SUPPORT

This invention was made with government support under grant numbers RO1 All 16723 and R21 A1097802 awarded by the National Institutes of Health. The government has certain rights in the invention.

### BACKGROUND

Schistosomiasis is a tropical parasitic disease (Hotez et al., 2008) caused by infections with flukes of the genus Schistosoma, affecting as many as 200 million individuals worldwide, with 779 million living at risk of infection (Steinmann et al., 2006; Colley at al. 2014). Schistosoma mansoni, S. haematobium and S. japonicum cause the largest public health burden (Gryseels, 2012; Colley et al., 2014). The disease gives rise to a persistent chronic disorder in endemic areas, resulting in common disabling complications such as anaemia, growth stunting, cognitive impairment, and decreased aerobic capacity (Terer et al., 2013; Colley et al., 2014). More severe disease manifestations include obstructive uropathy and bladder calcification (S. haematobium) and periportal hepatic fibrosis (S. mansoni and S. japonicum). An estimated 1.4 million disability-adjusted life years (DALYs) have been attributed to schistosomiasis using the most recent DALY metrics (Murray et al., 2018).

To reduce the chronic health burden, millions of school-aged children are treated each year in the framework of “preventive chemotherapy” programs with praziquantel (PZQ) (Knopp et al., 2013)

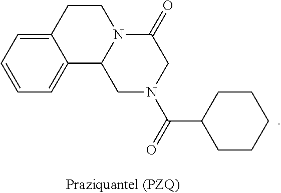 [SMILES: O=C(C1CCCCC1)N1CC(=O)N2CCc3ccccc3C2C1]

Praziquantel is the only drug available for treatment of this disease, but it is rapidly metabolized, rarely curative, and has little activity against juvenile schistosomula, the young developmental stage of the parasite (Utzinger et al., 2011; Olliaro et al., 2014; Bergquist et al., 2017). The high drug pressure from the widespread administration of PZQ could lead to problematic drug resistance (Melman et al., 2009), possibly due to upregulation of the schistosomal homologue of the P-glycoprotein drug transporter (Kasinathan and Greenberg, 2012). Should serious PZQ drug resistance arise, there are no viable alternatives to this drug (Keiser and Utzinger, 2012). Even so, drug discovery for schistosomiasis has languished (Horton, 2003; Geary et al., 2010; Caffrey and Secor, 2011), although several antischistosomal lead compounds (Njoroge et al., 2014; Mäder et al., 2018; Panic and Keiser, 2018; Caffrey et al., 2019) have been identified in recent years. Nonetheless, since no drug is currently undergoing clinical testing for schistosomiasis (Pedrique et al., 2013), a backup drug for PZQ will be not available in the near future.

Thus, there remains a need for antischistosomal compounds and methods of treating, inhibiting, and/or preventing parasitic diseases, including Schistosomiasis.

### SUMMARY

The disclosure provides compounds, or pharmaceutically acceptable salts thereof, having the structure of Formula (I):

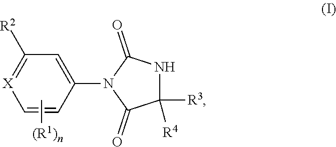

wherein

### X is CH, CF, or N;

each R¹ is F;
n is 0-3;
R² is selected from F, C, Br, CF₂H, CF₃, CFH₂, CF₂CH₃, CFHCFH₂, CH₂CF₃, and OCF₃; each of R³ and R⁴ is independently selected from CH₃, CD₃, CFH₂, CF₂H, CF₃, CF₂CH₃, CFHCFH₂, CH₂CF₃, and OCF₃; and when X is CF, R² is CF₃, and n is 0, R³ and R⁴ are not both CH₃.

The disclosure also provides pharmaceutical compositions comprising the disclosed compounds or pharmaceutically acceptable salts thereof and a pharmaceutically acceptable carrier or excipient.

The disclosure further provides methods of treating, inhibiting, and/or preventing a parasitic disease in a subject in need thereof, wherein the method comprises administering to said subject an effective amount of the disclosed compounds or pharmaceutically acceptable salts thereof.

### BRIEF DESCRIPTION OF THE DRAWINGS

FIG. 1 shows the plasma concentration of Compound IE in mice upon oral and IV administration.

FIG. 2 shows the plasma concentration of Compound IE in rats upon oral and IV administration.

FIG. 3 shows the plasma concentration of Compound IC in rats upon oral and IV administration.

FIG. 4 shows a comparison of plasma concentrations for Compounds IC and IE in male Sprague Dawley rats following IV and oral administration.

FIG. 5 shows the plasma profiles for Compound IE in male and female dogs after iv administration of 1 mg/kg. Each data point represents the mean of n=2 dogs.

FIG. 6 shows plasma profiles for Compound IE in male dogs after po administration of a single dose of 15, 75, and 150 mg/kg. Each data point represents the mean of n=2 dogs.

FIG. 7 shows plasma profiles for Compound IE in female dogs after po administration of a single dose of 15, 75, and 150 mg/kg. Each data point represents the mean of n=2 dogs.

FIG. 8 shows a comparison of plasma concentrations for Compound IE in female and male dogs after iv administration (1 mg/kg) or po (15 mg/kg).

### DETAILED DESCRIPTION

The present disclosure provides compounds useful for treating parasitic diseases. In various embodiments, the present disclosure provides aryl hydantoin compounds and pharmaceutical compositions thereof, and methods for the prophylaxis and treatment of Schistosomiasis.

The disclosed compounds provide several advantages over current treatment regimens, including those comprising “preventive chemotherapy” PZQ. Other conventional compounds used to treat Schistosomiasis include the conventional aryl hydantoins nilutamide and Ro-13-3978:

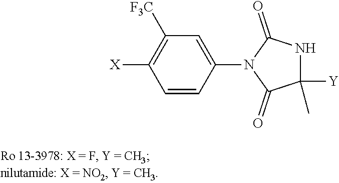

It is well established that conventional treatments suffer from numerous disadvantages, including undesirable pharmacokinetics (e.g., short half-life), poor activity profile (juvenile stage vs. adult stage), and unwanted side-effects (e.g., antiandrogenic effects). By way of example, PZQ is rapidly metabolized, rarely curative, and has little activity against juvenile schistosomula, the young developmental stage of the parasite. Furthermore, antiandrogenic effects have been observed in multiple-dose studies using male castrated rats (Bernauer et al., 1980). Moreover, Applicant has shown that both nilutamide and Ro 13-3978 undesirably inhibit dihydrotestosterone (DHT)-induced cell proliferation in the androgen-dependent LAPC4 prostate cancer cell line (Jones and Diamond, 2008) with respective IC₅₀ values of 0.52 and 11 μM. Moreover, nilutamide has very weak antischistosomal activity (Keiser et al., 2010).

In addition, conventional treatments suffer from poor activity against the juvenile stage of S. mansoni. Applicant and others have found that Ro 13-3978 is approximately 10 times less effective against the juvenile stage (ED₅₀=140 mg/kg) than the adult stage (ED₅₀=15 mg/kg) of S. mansoni in a mouse model (Link and Stohler, 1984; Keiser et al., 2015). In this same schistosome mouse model, PZQ is considerably less effective against adult S. mansoni, with reported ED₅₀ values ranging from 172 to 202 mg/kg and having no significant activity against juvenile stages of the parasite (Link and Stohler, 1984; Keiser et al., 2011).

In contrast to conventional compounds and treatment regimens known heretofore, the compounds of the present disclosure have improved pharmacokinetics (e.g., increased half-life), activity profiles (e.g., improved activity against the juvenile stage of S. mansoni), and exhibit diminished antiandrogenic effects.

Without wishing to be bound to any particular theory, it is believed that the activity of the disclosed compounds is mediated at least in part by a host effect. For example, despite the high in vivo antischistosomal efficacy of Ro 13-3978, Applicant has found that this aryl hydantoin at concentrations up to 170 μM had almost no effect on ex vivo adult S. mansoni (Keiser et al., 2015). As Ro 13-3978 forms only one minor and inactive Phase I metabolite AR40, active metabolites do not account for the significant difference between the in vitro and in vivo antischistosomal activity (Keiser et al., 2015; Wang et al., 2016) suggesting that the in vivo antischistosomal activity of Ro 13-3978 may be derived from host-mediated immune stimulation. For example, no significant changes were demonstrable via transcriptomic and ultraresolution microscopy when S. mansoni worms were exposed to Ro 13-3978 or Compound 1E (as disclosed herein) ex vivo. Moreover, it is believed that antibodies are required based on the fact that Ro 13-3978 effectively reduced S. mansoni worm burden in Foxn1^{nude} mice (Keiser et al., 2015), which have intact B-1 lymphocytes capable of producing evolutionarily conserved and T-independent immunoglobulins, but did not reduce worm burden (8% reduction, not statistically significant) in compound-treated NOD-SCID (Prkdc^{scid}) mice which lack all functional B cells as shown in Table 1.

TABLE 1
Worm burden reduction (WBR) in S. mansoni-infected
mice after single oral doses of Ro 13-3978 (100
mg/kg) and praziquantel (400 mg/kg).

| Mouse Type | Ro 13-3978 | Praziquantel (PZQ) |
|---|---|---|
| outbred NMRI | 95% | 96% |
| Foxn1^{nude} | 88% | ND |
| NOD-SCID (Prkdc^{scid}) | 8% | 83% |

In contrast, praziquantel reduced worm burden significantly in this strain by 83%. Single cell transcriptomic data conducted on the splenocytes of mice treated with Ro 13-3978 or compounds of the disclosure (e.g., Compound 1E) show transcriptional changes in reticulocytes, which is an outcome associated broadly with immune activation, and changes in subsets of innate and adaptive immune cells—most prominently, in neutrophils. Neutrophils are known innate immune protective cells against Schistosoma and work together with eosinophils in tissue clearance of worms.

### Compounds of the Disclosure

The disclosure provides a compound, or pharmaceutically acceptable salt thereof, having the structure of Formula (I):

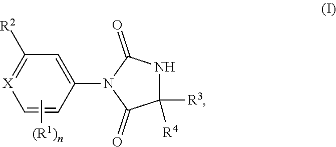

wherein

### X is CH, CF, or N;

each R¹ is F;
n is 0-3
R² is selected from F, C, Br, CF₂H, CF₃, CFH₂, CF₂CH₃, CFHCFH₂, CH₂CF₃, and OCF₃; each of R³ and R⁴ is independently selected from CH₃, CD₃, CFH₂, CF₂H, CF₃, CF₂CH₃, CFHCFH₂, CH₂CF₃, and OCF₃; and
when X is CF, R² is CF₃, and n is 0, R³ and R⁴ are not both CH₃.

### X

As described herein, X of compounds of Formula (I) can be CH, CF, or N. In some embodiments, X is CH. In some embodiments, X is N. In some embodiments, X is CF. Thus, in various embodiments, the ring comprising X of Formula (I) can have any of the following structures

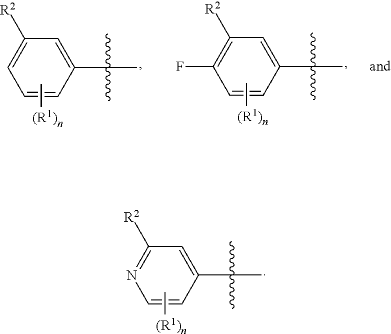

n

Compounds of the disclosure comprise the moiety (R¹)ₙ, wherein n is 0-3. In some embodiments, in conjunction with other embodiments herein, n is 0 such that the ring comprising X has the formula

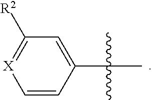

In some embodiments, in conjunction with other embodiments herein, n is 1. In some embodiments, in conjunction with other embodiments herein, n is 2. In some embodiments, in conjunction with other embodiments herein, n is 3.

In embodiments wherein n is 1 or 2, the relative positions of R¹ is not particularly limited. Thus, in various embodiments, the ring comprising X of Formula (I) can have any of the following structures:

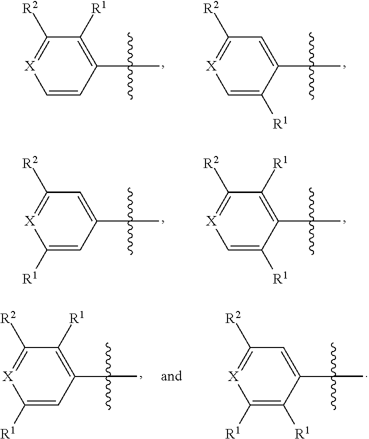

### R²

Compounds disclosed herein comprise R², wherein R² is selected from F, Cl, Br, CF₂H, CF₃, CFH₂, CF₂CH₃, CFHCFH₂, CH₂CF₃, and OCF₃. In some embodiments, in conjunction with other embodiments herein, R² is F, Cl, or Br. In some embodiments, in conjunction with other embodiments herein, R² is CF₃ or CFH₂. In some embodiments, in conjunction with other embodiments herein, R² is CF₂CH₃, CFHCFH₂, or CH₂CF₃. In some embodiments, in conjunction with other embodiments herein, R² is OCF₃. In some embodiments, in conjunction with other embodiments herein, R² is CF₃. Thus, in various embodiments, the ring comprising R² can have any of the following structures:

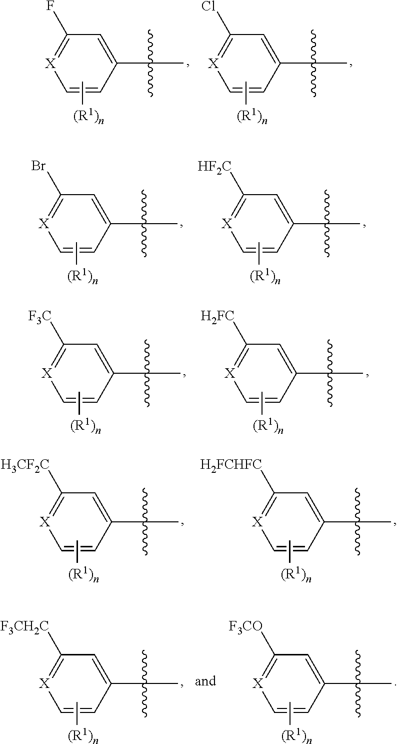

R³ and R⁴

Compounds disclosed herein comprise R³ and R⁴, wherein each of R³ and R⁴ is independently selected from CH₃, CD₃, CFH₂, CF₂H, CF₃, CF₂CH₃, CFHCFH₂, CH₂CF₃, and OCF₃. In some embodiments, each of R³ and R⁴ is independently selected from CH₃, CF₃ or CF₂H, and CD₃. In some embodiments, in conjunction with other embodiments herein, at least one of R³ or R⁴ is CH₃. In some embodiments, in conjunction with other embodiments herein, at least one of R³ or R⁴ is CF₃. In some embodiments, in conjunction with other embodiments herein, at least one of R³ or R⁴ is CF₂H. In some embodiments, in conjunction with other embodiments herein, at least one of R³ or R⁴ is CD₃.

### Compounds (IA)-(IF)

In some embodiments, in conjunction with other embodiments herein, the compound of Formula (I) is selected from Formulae (IA)-(IF) as shown herein:

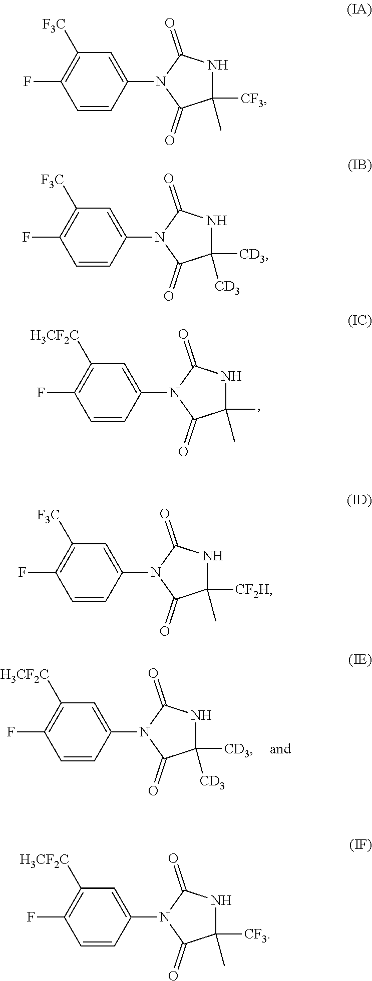

In some embodiments, the compound of Formula (I) is Formula (IE) or Formula (IF). In some embodiments, the compound of Formula (I) is Formula (IE). In some embodiments, the compound of Formula (I) is Formula (IF). In some embodiments, the compound of Formula (I) is Formula (IC).

### Pharmaceutically Acceptable Salts

The compounds disclosed herein can be in the form of a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other illustrative pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Pharmaceutical Compositions

The compounds described herein can be administered to a subject in a therapeutically effective amount (e.g., in an amount sufficient to prevent or relieve the symptoms of a parasitic disease). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

The methods can comprise administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

In some embodiments, the methods comprise administering a compound of Formula (I) at a dosage of 100 mg/kg, or 50 mg/kg, or 25 mg/kg, or 12.5 mg/kg, or 6.25 mg/kg, or 1 mg/kg.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

In some embodiments, the disclosure provides pharmaceutical compositions comprising a compound of Formula (I) as disclosed herein or a pharmaceutically acceptable salts thereof, and a pharmaceutically acceptable carrier or excipient. In some embodiments, the disclosed pharmaceutical compositions comprise a compound of Formula (IA), Formula (IB), Formula (IC), Formula (ID), Formula (IE), or a compound of Formula (IF). In some embodiments, the pharmaceutical compositions comprise a compound of Formula (IE) or (IF). In some embodiments, the pharmaceutical compositions comprise a compound of Formula (IE).

As used herein, the terms carrier or excipient are used interchangeably unless otherwise specified. Accordingly, a pharmaceutically acceptable carrier or excipient refers to any pharmaceutically acceptable additive, carrier, diluent, adjuvant, or other ingredient, other than the active pharmaceutical ingredient (API).

Suitable methods of administering a physiologically-acceptable composition, such as a pharmaceutical composition comprising the compounds disclosed herein (e.g., compounds of Formula I, Formula IA, Formula IB, Formula IC, Formula ID, Formula IE, Formula IF, or pharmaceutically acceptable salts thereof), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The pharmaceutical compositions used in the methods of the disclosure may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, PA, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus, the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As described herein, it is believed that the activity of the disclosed compounds may be due, at least in part, to a host immune response. Accordingly, in some embodiments, the disclosed pharmaceutical compositions are suitable for stimulating an immune response in a subject. Thus, in some embodiments the disclosed pharmaceutical compositions further comprise at least one vaccine antigen. Illustrative suitable vaccine antigens include, for example, Smp80, Smp28, Sm14, Sj23, Cathepsin B-like cysteine proteinase, and schistosome glutathione S-transferase P28GST.

### Methods of Use

In some embodiments, the disclosure provides methods of treating, inhibiting, and/or preventing a parasitic disease in a subject in need thereof, wherein the method comprises administering to said subject an effective amount of the disclosed compounds or pharmaceutically acceptable salt thereof. Parasitic based infections include but are not limited to schistosoma-based infections (e.g., Schistosomiasis). The compounds of the disclosure can be used by themselves or combined with other therapeutics. In some embodiments, a compound of Formula (I) is administered with at least one vaccine antigen.

As used herein, treating refers to reducing the severity of symptoms or effects of a parasitic disease. As used herein, inhibiting refers to slowing down the progression of a parasitic disease. As used herein, preventing an increase in the severity of symptoms. As used herein, preventing refers to reducing the chance of contracting a parasitic disease.

As used herein, the term “therapeutically effective amount” means an amount of a compound or combination of therapeutically active that ameliorates, attenuates or eliminates one or more symptoms of a particular disease or condition (e.g., parasitic disease), or prevents or delays the onset of one of more symptoms of a particular disease or condition.

As used herein, the terms “subject” and “patient” may be used interchangeably and mean animals, such as dogs, cats, cows, horses, and sheep (e.g., non-human animals) and humans. Particular subjects or patients are mammals (e.g., humans). The terms subject and patient include males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

In some embodiments, the disclosed methods comprise administering a compound selected from Compound of Formula (IA), Formula (IB), Formula (IC), Formula (ID), Formula (IE), and Formula (IF), or a pharmaceutically acceptable salt thereof. In some embodiments, the disclosed methods comprise administering Compound of Formula (IE) or Formula (IF), or a pharmaceutically acceptable salt thereof. In some embodiments, the disclosed methods comprise administering a Compound of Formula (IE) or a pharmaceutically acceptable salt thereof.

Illustrative suitable parasitic based infections which can be treated, inhibited, and/or prevented by administration of the disclosed compound include schistosoma-based infections such as, for example, Schistosomiasis. In some embodiments, the parasitic infection is Schistosomiasis.

The disclosed methods comprise administering a compound of Formula (I) using any suitable route of administration. Illustrative suitable routes of administration include, for example, parenterally, subcutaneously, orally, topically, pulmonarily, rectally, vaginally, intravenously, intraperitoneally, intrathecally, intracerbrally, epidurally, intramuscularly, intradermally, or intracarotidly. In some embodiments, a compound of Formula (I) is administered orally. In some embodiments, a compound of Formula (I) is administered intravenously.

The compositions of the present invention may be administered to a patient and may be conveniently formulated for administration with any pharmaceutically acceptable carrier(s).

Uses of the compounds disclosed herein in the preparation of a medicament for treating, inhibiting, and/or preventing parasitic diseases also are provided herein.

### Embodiments

The disclosure encompasses various embodiments as set forth below.

1. A compound, or pharmaceutically acceptable salt thereof, having the structure of Formula (I):

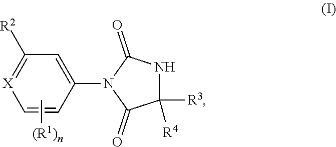

wherein

### X is CH, CF, or N;

each R¹ is F;
n is 0-3;
R² is selected from F, C, Br, CF₂H, CF₃, CFH₂, CF₂CH₃, CFHCFH₂, CH₂CF₃, and OCF₃;
each of R³ and R⁴ is independently selected from CH₃, CD₃, CFH₂, CF₂H, CF₃, CF₂CH₃, CFHCFH₂, CH₂CF₃, and OCF₃; and
when X is CF, R² is CF₃, and n is 0, R³ and R⁴ are not both CH₃.

2. The compound or pharmaceutically acceptable salt of embodiment 1, wherein X is CF.

3. The compound or pharmaceutically acceptable salt of embodiment 1 or 2, wherein n is 0.

4. The compound or pharmaceutically acceptable salt of any one of embodiments 1-3, wherein R² is CF₃.

5. The compound or pharmaceutically acceptable salt of any one of embodiments 1-4, wherein each of R³ and R⁴ is independently selected from CH₃, CF₃ or CF₂H, and CD₃.

6. The compound or pharmaceutically acceptable salt of any one of embodiments 1-5, wherein at least one of R³ or R⁴ is CH₃.

7. The compound or pharmaceutically acceptable salt of any one of embodiments 1-6, wherein at least one of R³ or R⁴ is CF₃.

8. The compound or pharmaceutically acceptable salt of any one of embodiments 1-7, wherein at least one of R³ or R⁴ is CF₂H.

9. The compound or pharmaceutically acceptable salt of any one of embodiments 1-8, wherein at least one of R³ or R⁴ is CD₃.

10. The compound or pharmaceutically acceptable salt of any one of embodiments 1-9, wherein the compound of Formula (I) is selected from

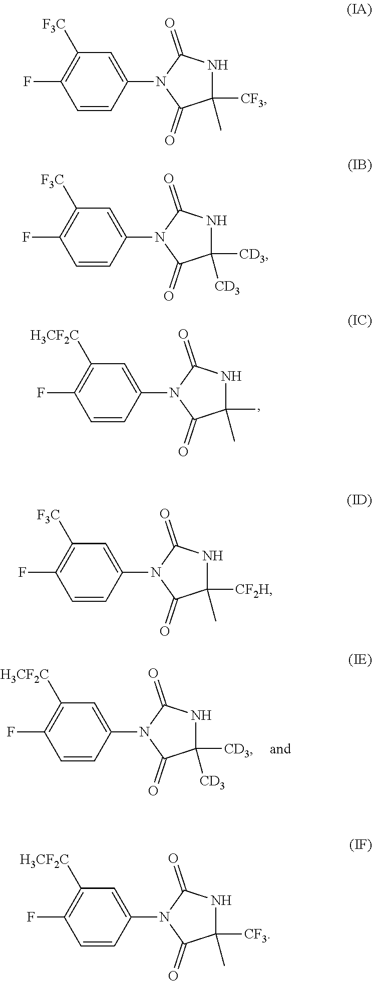

11. The compound or pharmaceutically acceptable salt of embodiment 10, wherein the compound of Formula (I) is Formula (IE) or Formula (IF).

12. The compound or pharmaceutically acceptable salt of embodiment 10 or 11, wherein the compound of Formula (I) is Formula (IE).

13. A pharmaceutical composition comprising a compound or pharmaceutically acceptable salt according to any one of embodiments 1-12 and a pharmaceutically acceptable carrier or excipient.

14. The pharmaceutical composition of embodiment 13, wherein the compound of Formula (I) is selected from a compound of Formula (IA), Formula (IB), Formula (IC), Formula (ID), Formula (IE), and Formula (IF).

15. The pharmaceutical composition of embodiment 13 or 14, wherein the compound of Formula (I) is a compound of Formula (IE) or Formula (IF).

16. The pharmaceutical composition of embodiment 15, wherein the compound of Formula (I) is Formula (IE).

17. The pharmaceutical composition of any one of embodiments 13-16, further comprising at least one vaccine antigen.

18. A method of treating, inhibiting, and/or preventing a parasitic disease in a subject in need thereof, wherein the method comprises administering to said subject an effective amount of a compound or pharmaceutically acceptable salt according to any one of embodiments 1-12.

19. The method of embodiment 18, wherein the compound of Formula (I) is selected from a compound of Formula (IA), Formula (IB), Formula (IC), Formula (ID), Formula (IE), and Formula (IF).

20. The method of embodiment 19, wherein the compound of Formula (I) is a compound of Formula (IE) or Formula (IF).

21. The method of embodiment 20, wherein the compound of Formula (I) is Formula (IE).

22. The method of any one of embodiments 18-21, wherein the parasitic disease is Schistosomiasis.

23. The method of any one of embodiments 18-22, wherein the compound of Formula (I) is administered orally.

24. The method of any one of embodiments 18-23, wherein the compound of Formula (I) is administered with at least one vaccine antigen.

25. The composition of any one of embodiments 13-17 for use in treating, inhibiting, and/or preventing a parasitic disease in a subject in need thereof.

26. The composition for use of embodiment 25, wherein the compound of Formula (I) is selected from a compound of Formula (IA), Formula (IB), Formula (IC), Formula (ID), Formula (IE), and Formula (IF).

27. The composition for use of embodiment 26, wherein the compound of Formula (I) is a compound of Formula (IE) or Formula (IF).

28. The composition for use of embodiment 27, wherein the compound of Formula (I) is a compound of Formula (IE).

29. The composition for use of any one of embodiments 25-28, wherein the disease is Schistosomiasis.

30. Use of the composition of any one of embodiments 13-17 for treating, inhibiting, and/or preventing a parasitic disease in a subject in need thereof.

31. The use of embodiment 35, wherein the compound of Formula (I) is selected from a compound of Formula (IA), Formula (IB), Formula (IC), Formula (ID), Formula (IE), and Formula (IF).

32. The use of embodiment 36, wherein the compound of Formula (I) is a compound of Formula (IE) or Formula (IF).

33. The use of embodiment 37, wherein the compound of Formula (I) is a compound of Formula (IE).

34. The use of any one of embodiments 35-38, wherein the parasitic disease is Schistosomiasis.

The foregoing summary is not intended to define every aspect of the disclosure, and additional aspects are described in other sections of the disclosure. The entire document is intended to be related as a unified disclosure, and it should be understood that all combinations of features described herein are contemplated, even if the combination of features are not found together in the same sentence, or paragraph, or section of this document.

In addition to the foregoing, the disclosure includes, as an additional aspect, all embodiments of the disclosure narrower in scope in any way than the variations specifically mentioned above. With respect to aspects of the disclosure described or claimed with “a” or “an,” these terms mean “one or more” unless context unambiguously requires a more restricted meaning. With respect to elements described as one or more within a set, all combinations within the set are contemplated as combination inventions. If aspects of the disclosure are described as “comprising” a feature, embodiments also are contemplated “consisting of” or “consisting essentially of” the feature.

Aspects of the disclosure described as methods of treatment should also be understood to include first or subsequent “medical use” aspects of the disclosure or “Swiss use” of compositions for the manufacture of a medicament for treatment of the same disease or condition.

Multiple embodiments are contemplated for combinations described herein. For example, some aspects of the disclosure that are described as a method of treatment (or medical use) combining two or more compounds or agents, whether administered separately (sequentially or simultaneously) or in combination (co-formulated or mixed). For each aspect described in this manner, the disclosure further includes a composition comprising the two or more compounds or agents co-formulated or in admixture with each other; and the disclosure further includes a kit or unit dose containing the two or more compounds/agents packaged together, but not in admixture. Optionally, such compositions, kits or doses further include one or more carriers in admixture with one or both agents or co-packaged for formulation prior to administration to a subject. The reverse also is true: some aspects of the disclosure are described herein as compositions useful for therapy and containing two or more therapeutic agents. Equivalent methods and uses are specifically contemplated.

Although the applicant(s) invented the full scope of the claims appended hereto, the claims appended hereto are not intended to encompass within their scope the prior art. Therefore, in the event that statutory or judicially recognized prior art within the scope of a claim is brought to the attention of the applicants by a Patent Office or other entity or individual, the applicant(s) reserve the right to exercise amendment rights under applicable patent laws to redefine the subject matter of such a claim to specifically exclude such prior art or obvious variations of statutory prior art from the scope of such a claim. Variations of the disclosure defined by such amended claims also are intended as aspects of the invention. Additional features and variations of the invention will be apparent to those skilled in the art from the entirety of this application, and all such features are intended as aspects of the disclosure.

The disclosure herein will be understood more readily by reference to the following examples, below.

### EXAMPLES

The following examples are provided for illustration and are not intended to limit the scope of the disclosure.

The following abbreviations are used herein: EA refers to ethyl acetate; DMA refers to dimethylacetamide; EtOH refers to ethanol; NaCN refers to sodium cyanide; NMR refers to nuclear magnetic resonance spectroscopy; rt refers to room temperature; DCM refers to dichloromethane; aq refers to aqueous; Et₂O refers to diethyl ether; HRMS refers to high resolution mass spectrometry; EI refers to electroionization;

The following examples describes illustrative procedures for preparing and evaluating Compounds (IA)-(IF).

General. Melting points are uncorrected. 1D ¹H and ¹³C NMR spectra were recorded on 400 and 500 MHz spectrometers using CDCl₃ or DMSO-d₆ as solvents. All chemical shifts are reported in parts per million (ppm) and are relative to internal (CH₃)₄Si (0 ppm) for ¹H and CDCl₃ (77.2 ppm) or DMSO-d₆ (39.5 ppm) for ¹³C NMR. El GC-MS data were obtained using a quadrapole mass spectrometer with 30 m DB-XLB type columns and a He flow rate of 1 mL/min. Silica gel (sg) particle size 32-63 μm was used for all flash column chromatography. Reported reaction temperatures are those of the oil bath. Combustion analysis confirmed that all target compounds have a purity of at least 95%.

Kinetic Solubility. Compounds in DMSO (10 mg/mL) were diluted into either pH 6.5 phosphate buffer or 0.01 M HCl (approx. pH 2.0) with the final DMSO concentration being 1%. Samples were then analysed via nephelometry to determine a solubility range (Bevan and Lloyd, 2000).

Partition Coefficient. Partition coefficient values (Log D) of the test compounds were estimated by correlation of their chromatographic retention properties against the characteristics of a series of standard compounds with known partition coefficient values using gradient HPLC (modification of a method reported by Lombardo et al., 2001).

Plasma Protein Binding. Plasma protein binding values of the test compounds were estimated by correlation of their chromatographic retention properties on a human albumin column against the characteristics of a series of standard compounds with known protein binding values. The method employed is a gradient HPLC based derivation of the method developed by Valko et al. (2003).

In Vitro Metabolic Stability. As described by Charman et al. (2020), metabolic stability assays were performed by incubating test compounds in liver microsomes at 37° C. and 0.4 mg/mL protein concentration. The metabolic reaction was initiated by the addition of an NADPH-regenerating system and quenched at various time points over a 60 min incubation period by the addition of acetonitrile containing diazepam as internal standard. Control samples (containing no NADPH) were included (and quenched at 2, 30 and 60 min) to monitor for potential degradation in the absence of cofactor.

Mouse Exposure Studies. The systemic exposure of the aryl hydantoins was studied in non-fasted male Swiss outbred mice weighing 25-33 g. Mice had access to food and water ad libitum throughout the pre- and post-dose sampling period. Formulations were prepared by dispersing the aryl hydantoins in Tween 80 and then adding ethanol and Milli-Q water (final composition 7% v/v Tween 80, 3% v/v ethanol). Following vortexing and sonication, samples of compounds formed either a uniform suspension or a colorless solution. Compound formulations were mixed by inverting the tubes prior to drawing each dosing volume. All animals were dosed orally by gavage (10 mL/kg dose volume) within 1 h of formulation preparation. Following administration, blood samples were collected from 0.25-48 h post-dose (n=2 mice per time point). A maximum of two samples were obtained from each mouse, with samples being taken either via submandibular bleed (approximately 120 μL; conscious sampling) or terminal cardiac puncture (0.6 mL; while mice were anaesthetised using inhaled Isoflurane). No urine samples were collected as mice were housed in bedded cages during the study. Blood was collected directly into polypropylene Eppendorf tubes containing heparin as anticoagulant and stabilisation cocktail (containing Complete® (a protease inhibitor cocktail), potassium fluoride and EDTA) to minimise the potential for ex vivo degradation of the aryl hydantoins in blood/plasma samples. Once collected, blood samples were centrifuged immediately, supernatant plasma was removed, and stored at −80° C. until analysis by LCMS. Plasma concentration-time data were analysed using noncompartmental methods (PKSolver Version 2.0).

Antischistosomal Screen. In vivo antischistosomal properties were assessed by measuring worm burden reduction (WBR) in S. mansoni-infected mice at either 100 or 50 mg/kg. As described by Lombardo et al. (2019), cercariae of S. mansoni were obtained from infected Biomphalaria glabrata. By way of example, NMRI mice were infected subcutaneously with approximately 100 S. mansoni cercariae. At 49 d after infection, groups of four mice were treated with single 100 mg/kg oral doses of compounds in a 7% (v/v) Tween 80% and 3% (v/v) ethanol vehicle (10 mL/kg). Untreated mice (n=8) served as controls. At 21 d post-treatment, animals were killed by the CO₂ method and dissected. Worms were removed by picking, then sexed and counted.

Androgen-Dependent Cell-based Assay. As described by Jones and Diamond (2008), LAPC4 cells were cultured in phenol red free RPMI 1640 media supplemented with antibiotics and 10% FBS. For all transfections, pools of cells were transfected using Lipofectamine Plus (Invitrogen) with PSA-luciferase (Bolton et al. 2007) and pRL-SV40 (Promega) as a normalization control. The following day, the cells were re-plated, 0.3 nM DHT and test compounds were added, and 24 h later luciferase production was measured (Dual luciferase assay kit; Promega), normalizing the firefly signal to the renilla signal. Mean-effect plots (log[compound] vs. log[fractional effect]) were generated to determine the IC₅₀ values for each test compound or combinations of test compounds at constant ratios. In this assay, the antiandrogen hydantoin drug nilutamide has an IC₅₀ of 0.52 μM (Wang et al., 2016).

### Example 1. Synthesis of Compounds

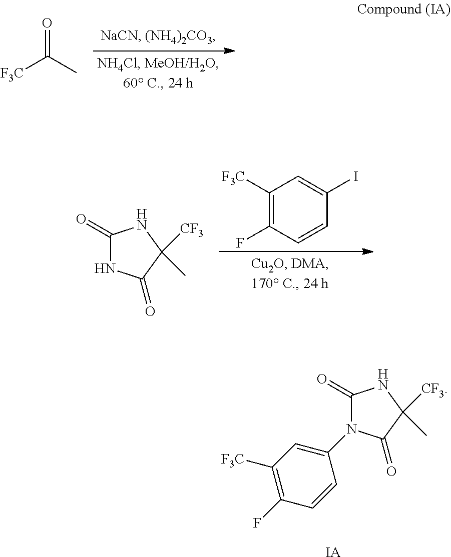

3-(4-Fluoro-3-(trifluoromethyl)phenyl)-5-methyl-5-(trifluoromethyl)imidazolidine-2,4-dione (IA). Step 1. To a solution of 1,1,1-trifluoropropan-2-one (1.520 g, 13.6 mmol), (NH₄)₂CO₃ (2.784 g, 29 mmol), and NH₄Cl (2.894 g, 54.1 mmol) in water (10 mL) and MeOH (10 mL) was added NaCN (1.329 g, 27.1 mmol) dissolved in a minimal amount of water. After it was heated at 60° C. for 24 h, 10% aq. HCl was added until a pH of 6 was reached. The solution was concentrated and was extracted with ethyl acetate (4×30 mL). The organic layer was dried with brine and MgSO₄ and then the solvent was evaporated to yield 5-methyl-5-(trifluoromethyl)imidazolidine-2,4-dione as a crude red semi-solid (1.200 g, 49%) which was not further purified. ¹H NMR (400 MHz, DMSO-d₆) δ1.53 (m, 3H), 8.90 (s, 1H), 11.33 (s, 1H). Step 2. To a solution of 5-methyl-5-(trifluoromethyl)imidazolidine-2,4-dione (834 mg, 4.6 mmol) and Cu₂O (380 mg, 2.7 mmol) in DMA (10 mL) was added 1-fluoro-4-iodo-2-(trifluoromethyl)benzene (698 mg, 5.9 mmol). The mixture heated to 170° C. for 48 h followed by solvent evaporation in vacuo. The residue was purified by sg chromatography (hexane to 1:1 EA:hexane) and recrystallization (ether:hexane, 1:10) to afford the product as a white crystalline solid. mp 112-115° C. ¹H NMR (400 MHz, DMSO-d₆) δ 1.72 (s, 3H), 7.70 (t, J=9.7 Hz, 1H), 7.82 (m, 1H), 7.92 (dd, J=2.5, 6.5 Hz, 1H), 9.68 (s, 1H). ¹³C NMR (125 MHz, DMSO-d₆) δ 16.34, 62.67 (q, J=29.2 Hz), 117.07 (q, J=15.7 Hz), 118.20 (d, J=21.7 Hz), 121.65 (d, J=157.3 Hz), 123.87 (d, J=162.2 Hz), 125.86 (d, J=5.5 Hz), 127.78 (d, J=3.5 Hz), 133.77 (d, J=9.5 Hz), 154.09, 158.08 (d, J=255.9 Hz), 167.81. Anal. Calcd for C₁₂H₇F₇N₂O₂: C, 41.88; H, 2.05; N, 8.14. Found: C, 41.69; H, 2.14; H, 8.21.

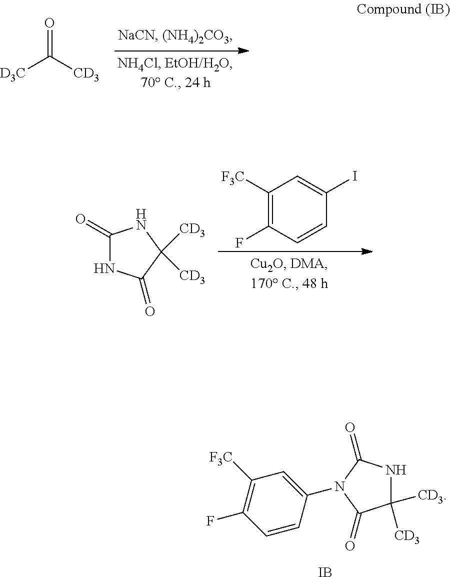

3-(4-Fluoro-3-(trifluoromethyl)phenyl)-5,5-bis(methyl-d3)imidazolidine-2,4-dione (IB). Step 1. In a pressure flask, to a solution of deuterated acetone (1.509 g, 23.5 mmol) and (NH₄)₂CO₃ (4.454 g, 46.4 mmol) in water (10 mL) and EtOH (10 mL) was added NaCN (2.261 g, 46.1 mmol) dissolved in a minimal amount of water. The vessel was sealed and heated at 70° C. for 24 h. The reaction was cooled to rt and NH₄Cl (2.5 g, 46.7 mmol) was added. The mixture was sealed again and heated to 70° C. for an additional 24 h. 10% HCl was then added until a pH of 6 was reached. The solution was concentrated and was extracted with EA (4×30 mL). The organic layer was dried with brine and MgSO₄ and the solvent was evaporated in vacuo to yield 5,5-bis(methyl-d3)imidazolidine-2,4-dione as a white microcrystalline solid (2.610 g, 83%) which was not further purified. ¹H NMR (400 MHz, DMSO-d₆) δ 7.92 (s, 1H), 10.53 (s, 1H). Step 2. To a solution of 5,5-bis(methyl-d3)imidazolidine-2,4-dione (1.420 g, 10.6 mmol) and Cu₂0 (899 mg, 6.3 mmol) in DMA (25 mL) was added 1-fluoro-4-iodo-2-(trifluoromethyl)benzene (3.657 g, 12.6 mmol) in a pressure flask. The mixture was heated to 170° C. for 48 h followed by solvent evaporation in vacuo. The residue was purified by chromatography (hexane to EA) and recrystallization (ether/hexane, 1:10) to afford the product as a white microcrystalline solid. mp 112-115° C. ¹H NMR (500 MHz, DMSO-d₆) δ 7.66 (t, J=9.7 Hz, 1H), 7.79-7.84 (m, 1H), 7.90 (dd, J=2.5, 6.7 Hz, 1H), 8.64 (s, 1H). ¹³C NMR (125 MHz, DMSO-d₆) δ 23.70, 57.64, 116.69 (dd, J=13.5, 32.8 Hz), 117.71 (d, J=21.7 Hz), 122.23 (d, J=271.9 Hz), 125.70 (d, J=4.8 Hz), 128.90 (d, J=3.2 Hz), 133.59 (d. J=9.1 Hz), 153.70, 157.55 (d, J=255.4 Hz), 176.15. HRMS (El-Ion trap) m/z: [M+] Calcd for C₁₂H₄D₆F₄N₂O₂ 297.1128, found 297.1105.

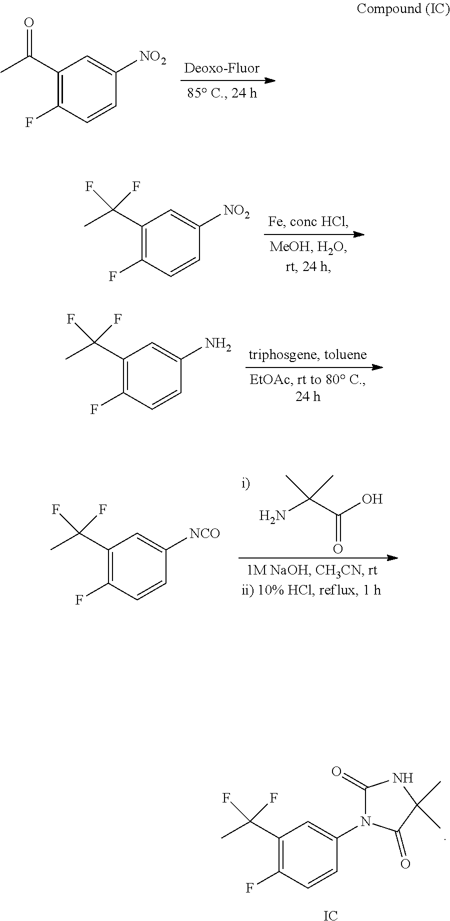

3-(3-(1,1-difluoroethyl)-4-fluorophenyl)-5,5-dimethylimidazolidine-2,4-dione (IC). Step 1. Using a modified method of Vandyck et al. (2014), Deoxo-Fluor (7.360 g, 33.3 mmol) was added dropwise to a pressure vessel charged with 1-(2-fluoro-5-nitrophenyl)ethan-1-one (3.011 g, 16.4 mmol) at rt with stirring. The vessel was sealed and the mixture was heated to 85° C. for 24 h. After cooling to rt, sat. NaHCO₃ (20 mL) was added slowly to the reaction mixture. After CO₂ evolution ceased, DCM was added (20 mL) and the organic layer was separated. The aq. layer was extracted with DCM (2×20 mL) and the combined organic layers were washed with brine and dried with MgSO₄. Solvent was removed in vacuo to yield 2-(1,1-difluoroethyl)-1-fluoro-4-nitrobenzene as a crude red oil which was used in the next step without further purification. ¹H NMR (400 MHz, CDCl₃) δ 2.04 (td, J=1.1, 18.6 Hz, 3H), 7.32 (t, J=9.3 Hz, 1H), 8.35 (ddd, J=2.9, 4.1, 9.0 Hz, 1H), 8.49 (dd, J=2.9, 6.3 Hz, 1H). Step 2. To a solution of crude 2-(1,1-difluoroethyl)-1-fluoro-4-nitrobenzene (2.880 g, 14.0 mmol), in CH₃OH (20 mL) and H₂O (10 mL), was added conc. HCl (10 mL) dropwise with stirring. Fe powder (4 g, 71.6 mmol) was added in aliquots and the reaction was stirred for 24 h. The mixture was filtered through Celite and the filtrate was basified to pH 10 with sat. K₂CO₃. The mixture was washed with DCM (25 mL) and the organic layer was separated. The aq. layer was extracted with DCM (2×20 mL) and the combined organic layers were washed with brine and dried with MgSO₄ to give 3-(1,1-difluoroethyl)-4-fluoroaniline as a crude yellow oil that was used without further purification. ¹H NMR (600 MHz, CDCl₃) δ 1.96 (t, J=18.6 Hz, 3H), 3.63 (s, 2H), 6.62-6.70 (m, 1H), 6.81 (dd, J=2.7, 6.0 Hz, 1H), 6.90 (t, J=9.6 Hz, 1H). ¹³C NMR (150 MHz, CDCl₃) δ 25.16 (td, J=3.7, 28.9 Hz), 112.36 (td, J=2.2, 7.8 Hz), 117.05 (d, J=23.1 Hz), 117.33 (d, J=7.7 Hz), 120.12 (td, J=2.1, 239.8 Hz), 125.56 (td, J=13.4, 27.1 Hz), 142.39 (d, J=2.2 Hz), 152.78 (d, J=241.1 Hz). Step 3. To a solution of triphosgene (1.469 g, 5.0 mmol) in toluene (10 mL) at 0° C. was added slowly a solution of 3-(1,1-difluoroethyl)-4-fluoroaniline (1.734 g, 9.9 mmol) in EA (10 mL). After the reaction was allowed to warm to rt, the reaction mixture was heated to 80° C. for 16 h. The reaction was cooled to rt and the solvent was removed to yield 2-(1,1-difluoroethyl)-1-fluoro-4-isocyanatobenzene as a yellow oil which was used for the next step without further purification. Step 4. To a stirring solution of 2-aminoisobutyric acid (1.253 g, 12.2 mmol) in 1 M NaOH (20 mL) at 0° C., was added crude 2-(1,1-difluoroethyl)-1-fluoro-4-isocyanatobenzene dropwise in CH₃CN (10 mL). After it was stirred at rt for 16 h, the reaction mixture was concentrated in vacuo and the aq. layer washed with EA (30 mL). The aq. layer was then concentrated and the pH was adjusted to 1 with 10% aq. HCl. The solution was heated for 12 h with stirring. The reaction was cooled to rt and the aq. layer was washed with EA (3×20 mL). The combined organic layers were washed with brine and dried with MgSO₄. Solvent removal in vacuo gave a brown residue which was purified with sg chromatography (hexane to 1:1 hexane:EA) and crystallized from Et₂O and hexane to give a white solid (531 mg, 19%). mp 109-110° C. ¹H NMR (400 MHz, DMSO) δ 1.41 (s, 6H), 2.03 (t, J=19.1 Hz, 3H), 7.49 (t, J=9.8 Hz, 1H), 7.55-7.73 (m, 2H), 8.62 (s, 1H). ¹³C NMR (125 MHz, DMSO) δ 24.41, 24.43, 24.65, 24.85, 24.87, 57.87, 117.17 (d, J=30.0 Hz), 120.29 (t, J=237.5 Hz), 124.56 (td, J=27.9, 13.7 Hz), 125.10 (td, J=6.9, 3.2 Hz), 128.52 (d, J=3.1H), 131.03 (d, J=9.2 Hz), 153.89, 157.65 (d, J=249.9 Hz), 176.24. Anal. Calcd for C₁₃H₁₃F₃N₂O₂: C, 54.55; H, 4.58; N, 9.79. Found: C, 54.57; H, 4.70; N, 9.87.

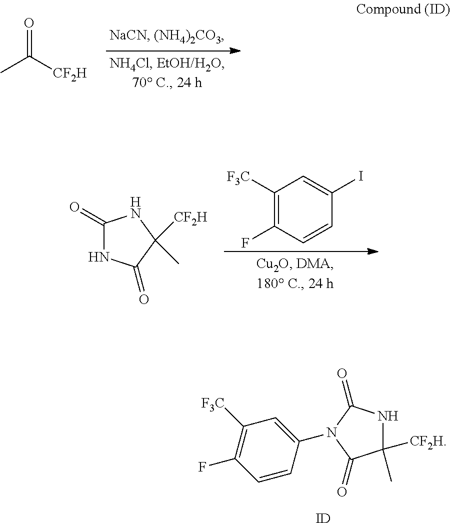

5-(Difluoromethyl)-3-(4-fluoro-3-(trifluoromethyl)phenyl)-5-methylimidazolidine-2,4-dione (ID). Step 1. To a pressure flask was added 1,1-difluoropropan-2-one (2.375 g, 25.3 mmol), (NH₄)₂CO₃ (4.9 g, 51.0 mmol), and NH₄Cl (2.770 g, 51.8 mmol). Water (10 mL) and EtOH (10 mL) were added and the solution was stirred for 10 min. NaCN (2.598 g, 53.0 mmol) was dissolved in a minimal amount of water and added to the mixture. The vessel was sealed and heated at 70° C. for 24 h. The reaction was cooled and 10% HCl was added until a pH of 1 was reached. The solution was concentrated in vacuo and was extracted with EA (3×30 mL). The organic layer was washed with brine and dried with MgSO₄ before solvent removal in vacuo to yield 5-(difluoromethyl)-5-methylimidazolidine-2,4-dione as an orange microcrystalline solid (2.982 g, 72%) which was not further purified. ¹H NMR (400 MHz, DMSO) Q 1.37 (s, 3H), 6.15 (t, J=54.6 Hz, 1H), 8.47 (s, 1H), 11.01 (s, 1H). Step 2. A stirred solution of 1-fluoro-4-iodo-2-(trifluoromethyl)benzene (3.411 g, 11.8 mmol), 5-(difluoromethyl)-5-methylimidazolidine-2,4-dione (1.684 g, 10.3 mmol), Cu₂0 (753 mg, 5.3 mmol), and DMA (10 mL) in a pressure vessel and heated to 180° C. for 24 h. The solvent was removed in vacuo and purification by sg chromatography (hexane to 1:1 hexane:EA) gave a crude solid which was recrystallized (1:5 Et₂O:hexane) to give a white microcrystalline solid (1.5 g, 45%). mp 110-112° C. ¹H NMR (500 MHz, DMSO) δ 1.54 (s, 3H), 6.34 (t, J=54.7 Hz, 1H), 7.69 (t, J=9.7 Hz, 1H), 7.75-7.84 (m, 1H), 7.88 (dd, J=2.5, 6.6 Hz, 1H), 9.24 (s, 1H). ¹³C NMR (125 MHz, DMSO) δ 16.00, 62.34 (t, J=21.4 Hz), 114.06 (t, J=246.2 Hz), 116.85 (dd, J=13.4, 33.0 Hz), 117.98 (d, J=21.8 Hz), 122.07 (d, J=272.4 Hz), 125.59 (d, J=4.8 Hz), 128.11 (d, J=3.5 Hz), 133.55 (d, J=9.4 Hz), 154.24, 157.77 (d, J=257.0 Hz), 169.78-170.52 (m). Anal. Calcd for C₁₂H₈F₆N₂O₂·0.5H₂O: C, 43.00; H, 2.71; N, 8.36. Found: C, 43.00; H, 2.36; N, 8.43.

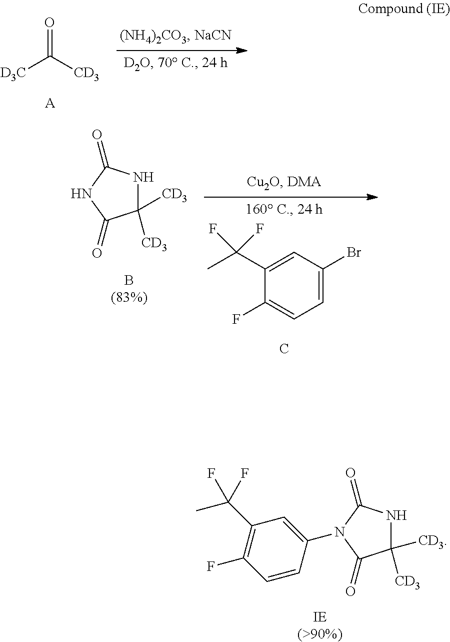

3-(3-(1,1-difluoroethyl)-4-fluorophenyl)-5,5-bis(methyl-d₃)imidazolidine-2,4-dione (IE). Step 1. To a solution of deuterated acetone (A) (1.509 g, 23.5 mmol) and (NH₄)₂CO₃ (4.454 g, 46.4 mmol) in D₂0 (10 mL) in a pressure flask was added NaCN (2.261 g, 46.1 mmol). The vessel was sealed and heated at 70° C. for 24 h. After cooling, conc. HCl was added until a pH of 6 was reached. The solution was concentrated in vacuo and the reaction residue was extracted with EA (4×30 mL). The organic layer was extracted with brine (approx. 50 mL) and then dried over MgSO₄. The solvent was removed in vacuo to yield 5,5-bis(methyl-d₃)imidazolidine-2,4-dione (B) as a white microcrystalline solid (2.610 g, 83%) which was used directly in the next step. ¹H NMR (400 MHz, DMSO-d₆) δ 7.92 (s, 1H), 10.53 (s, 1H). Step 2. A solution of B (4.769 g, 35.5 mmol) and 2-(1,1-difluoroethyl)-1-fluoro-4-bromobenzene (C) (6.438 g, 26.9 mmol), Cu₂0 (2.095 mg, 14.6 mmol), and DMA (4 mL) was stirred and heated to 160° C. for 24 h under Ar. The majority of solvent was removed in vacuo and the residue was digested in EA (20 mL). The mixture was filtered through a layered short column of celite and silica gel which was eluted with EA (3×30 mL).¹ The combined organic layers were washed with water (40 mL), 5 M NH₄0H (30 mL), and brine (50 mL). The organic layer was dried with Na₂SO₄. The solvent was removed and the residue was crystallized from 1:10 Et₂O:hexane (approx. 100 mL), filtered and washed with hexane (2×30 mL) to give a white solid (7.224 g, 92%). mp 110-111° C.; ¹H NMR (500 MHz, DMSO) δ 2.03 (t, J=19.1 Hz, 3H), 7.49 (t, J=9.7 Hz, 1H), 7.55-7.72 (m, 2H), 8.59 (s, 1H). ¹³C NMR (125 MHz, DMSO) δ 23.85 (m), 24.62 (td, J=2.9, 27.7 Hz), 57.55, 117.15 (d, J=23.1 Hz), 120.27 (t, J=238.1 Hz), 124.54 (d, J=13.4 Hz), 124.89-125.37 (m), 128.50, 131.02 (d, J=9.3 Hz), 153.90, 157.64 (d, J=251.0 Hz), 176.28.

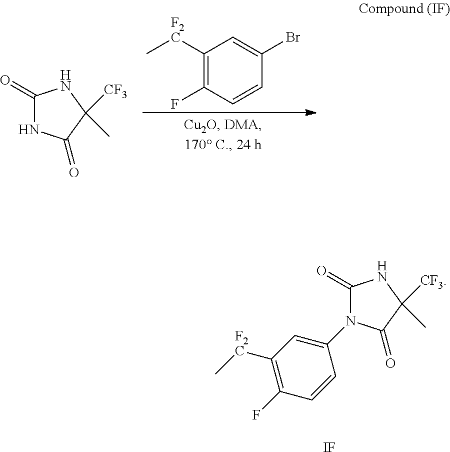 [SMILES: CC1(C(F)(F)F)NC(=O)NC1=O ; CC(F)(F)c1cc(Br)ccc1F ; CC(F)(F)c1cc(N2C(=O)NC(C)(C(F)(F)F)C2=O)ccc1F ; "IF"]

3-(4-Fluoro-3-(trifluoromethyl)phenyl)-5-methyl-5-(trifluoromethyl)imidazolidine-2,4-dione (IF). To a stirred solution of 5-methyl-5-(trifluoromethyl)imidazolidine-2,4-dione (2.033 g, 11.2 mmol) and Cu₂0 (731 mg, 5.1 mmol) in DMA (10 mL) was added 4-bromo-2-(1,1-difluoroethyl)-1-fluorobenzene (1.0 g, 4.2 mmol). The vessel was sealed and the mixture heated to 170° C. for 24 h before then solvent evaporation in vacuo. The residue was purified by chromatography (hexane to 1:1 EA:hexane) and recrystallization (ether:hexane, 1:10) to afford the product as a yellow crystalline solid. mp 120-122° C. ¹H NMR (500 MHz, DMSO) δ 1.71 (s, 3H), 2.03 (t, J=19.2 Hz, 3H), 7.53 (t, J=9.7 Hz, 1H), 7.57-7.63 (m, 1H), 7.65 (dd, J=2.0, 6.8 Hz, 1H), 9.63 (s, 1H). ¹³C NMR (125 MHz, DMSO) δ 16.32, 24.60 (td, J=2.8, 27.8 Hz), 62.61 (q, J=29.2 Hz), 117.61 (d, J=23.3 Hz), 120.21 (t, J=240.8 Hz), 123.47 (q, J=284.3 Hz), 124.84 (dd, J=13.7, 27.8 Hz), 125.07-125.59 (m), 127.47 (d, J=3.1 Hz), 131.16 (d, J=9.4 Hz), 154.29, 158.14 (d, J=252.2 Hz), 167.93. Anal. Calcd for C₁₃H₁₀F₆N₂O₂: C, 45.89; H, 2.96; N, 8.23. Found: C, 46.03; H, 3.00; N, 8.45.

### Example 2. Physicochemical, In Vitro ADME, and In Vivo Antischistosomal Activity

The physicochemical, in vitro ADME properties, and in vivo antischistosomal activity of Compounds (IA)-(IF) in mice were evaluated as described herein and summarized in Table 2.

TABLE 2
Compounds IA-IF at 100 mg/kg PO in mice

| | | | h/m CLᵢₙₜ | | | | S. mansoni A/J¹ |
|---|---|---|---|---|---|---|---|
| | | Sol_{2.0}/Sol_{6.5} | (μL/min/mg | Cₘₐₓ | AUC₀₋ₗₐₛₜ | LAPC4 IC₅₀ | ED₅₀ |
| Compound | gLogD_{7.4} | (μg/mL) | protein) | (μg/mL) | (μg/mL · h) | (μM) | (mg/kg) |
| Ro 13-3978 | 2.7 | >100/>100 | 8/<7 | 36 | 795 | 11 | 15/140 |
| IA | 3.5 | 25-50/25-50 | <7/<7 | 28 | 966 | 5.2 | 14/70 |
| IB | 2.7 | >100/>100 | <7/<7 | 43 | 1815 | 13 | 7.3/73 |
| IC | 2.6 | >100/>100 | 7/<7 | 40 | 1561 | >100 | 20/23 |
| ID | 3.2 | 50-100/50-100 | <7/<7 | 42 | 1646 | 4.2 | 9.0/88 |
| IE | 2.5 | >100/>100 | <7/<7 | 41 | 1799 | >100 | 7.1/16 |
| IF | 3.4 | 50-100/50-100 | <7/<7 | 34 | 1286 | 2.9 | 1.2/8.9 |

¹A/J is adult/juvenile

As shown in Table 2, the physicochemical and in vitro ADME properties of these compounds including Log D_{7.4} values of 2.7-3.5, aqueous kinetic solubilities of 25 to >100 μg/mL, estimated plasma protein binding (cPPB) values of 33-92%, and human (h) and mouse (m) microsomal in vitro CLᵢₙₜ values ranging from <7 to 8 μL/min/mg protein are associated with excellent pharmacokinetic profiles (see 100 mg/kg po Cₘₐₓ and AUC₀₋ₗₐₛₜ data). The superior pharmacokinetic properties of these compounds translated into promising antischistosomal efficacy; they all had substantially better single oral dose ED₅₀ values than Ro 13-3978 against adult (A) and juvenile (J) S. mansoni in a mouse model. These compounds also had low cytotoxicity. For example, none of these had measurable cytotoxicity at concentrations up to 50 μM against the human foreskin fibroblast (HFF), kidney (HEK293), hepatocyte (HC04), and B lymphocyte (RAJI) cell lines.

In particular, Compound IE replaces the hydrogen atoms of the gem-dimethyl substructure with deuteriums to slow Phase I metabolism and swapping the trifluoromethyl substituent with a difluoroethyl to abolish antiandrogenic effects (LAPC4 IC₅₀>100 μM). Compound IF replaces one of the gem-dimethyl groups of Ro 13-3978 with a trifluoromethyl. As the data in Table 2 demonstrates, both Compounds IE and IF have excellent in vitro ADME profiles and high in vivo antischistosomal activity, although the slightly more hydrophobic and less soluble Compound IF exhibited antiandrogenic activity (LAPC4 IC₅₀ 2.9 μM). Thus, a 5-trifluoromethyl group seems to override the antiandrogenic-weakening effect of a 3-difluoroethyl aryl substituent.

Following IV dosing at 1 mg/kg to mice, Ro 13-3978 and Compound IE had similar volumes of distribution of 1.88 and 1.59 L/kg, but very different apparent half-lives of 9.5 and 30 h; this can be attributed in part to the 3-fold higher blood clearance of 1.75 vs. 0.59 mL/min/kg for Ro 13-3978 and Compound IE, respectively. The superior pharmacokinetic properties of Compound IE translated into substantially better antischistosomal efficacy than Ro 13-3978-single oral dose ED₅₀ values of 7.1 and 16 mg/kg against adult (A) and juvenile (J) S. mansoni in a mouse model.

The photostability of Compound IE at 500 μg/mL concentration in 50% aq. MeOH also was evaluated under simulated confirmatory and forced degradation sunlight conditions. Compound IE showed minimal (<3%) loss of parent compound under all the light dose conditions and no degradation peaks were observed.

The chemical stability of Compound IE at 500 μg/mL in acid (0.01 M and 0.1 M HCl), base (0.01 M and 0.1 M NaOH) was evaluated for 48 h. Samples were neutralised and diluted to a nominal concentration of 100 μg/mL in 50% aq MeOH. Compound IE showed minimal loss of parent compound under both acidic conditions up to 48 h and no degradation peaks were observed in any of the samples. In contrast, Compound IE demonstrated significant loss of parent compound after just 2 min under both basic conditions. A single degradation peak appeared in all the basic samples corresponding to the urea carboxylic acid hydrolysis product.

Compound IE demonstrated no significant loss of parent compound under 15% H₂O₂ incubation conditions, but a single small peak (unidentified product) appeared after 48 h with a peak area of approximately 10% relative to Compound IE.

### Example 3—Further Studies of Compound IE and Compound IC

The activity of Compound IE was further evaluated as described herein. The results are summarized in Table 3.

TABLE 3
Summary of Properties of Compound IE
Physical Properties

| | |
|---|---|
| MW | 292.3 |
| Polar surface area | 49.4 Å |
| H-bond D/A | 1/2 |
| pKa (calc) | none |
| Log_{D7.4} (shake flask) | 1.9 |

Biorelevant Solubility

| | | Solubility |
|---|---|---|
| Media | pH | (μg/mL) |
| FaSSGF^{a} | 1.6 | 792 |
| FaSSIF (V2)^{b} | 6.5 | 940 |
| FeSSIF (V2) b | 5.8 | 1388 |
| PBS^{b} | 7.4 | 856 |

| | CLᵢₙₜ | CLᵢₙₜ |
|---|---|---|
| Species | (μL/min/mg) | (μL/min/10⁶) |
| Human | | <2 |
| Dog | | <2 |
| Rat | | <2 |
| Mouse | | <2 |

Permeability

| | |
|---|---|
| Apical-basolateral | 64.1 × 10⁻⁶ |
| Basolateral-apical | 57.8 × 10⁻⁶ |

Protein binding, B:P

| matrix | % bound | B:P |
|---|---|---|
| human plasma | 63 | 0.78 |
| Dog plasma | 68 | Not assessed |
| Rat plasma | 58 | 1.0 |
| Mouse plasma | 54 | 1.0 |
| HLM | 0 | — |

CYP inhibition

| CYP | IC₅₀ (μm) |
|---|---|
| 1A2 | >20 |
| 2B6 | >20 |
| 2C8 | >20 |
| 2C9 | >20 |
| 2C19 | >20 |
| 2D6 | >20 |
| 3A4* (tes/mid) | >20 |

Compound IE at a single 50 mg/kg oral dose completely cleared S. haematobium infections in hamsters, and at a single 25 mg/kg oral dose, also completely cleared S. japonicum infections in hamsters (ED₅₀ of 8.5 mg/kg) and mice (ED₅₀ of 6.7 mg/kg). Data not shown.

As previously noted, Compound IE did not inhibit DHT-induced cell proliferation in the androgen-dependent LAPC4 prostate cancer cell line at concentrations up to 100 μM. As an additional measure of androgen-receptor antagonist activity, the levels of DHT-induced (KLK3, FKBP5) and DHT-repressed (ZFP36L1) transcripts in LAPC4 cells treated with nilutamide and Compound IE were quantified. As expected, nilutamide inhibited the DHT-induced increase of KLK3 and FKBP5 and prevented the repression of ZFP36L1. In contrast, Compound IE had no effect at concentrations up to 10 μM.

Compound IE also exhibited good solubility across a range of biorelevant media including fasted (FaSSIF-V2; pH 6.5, 5 h at 37° C.) and fed (FeSSIF-V2; pH 5.8, 5 h at 37° C.) state simulated intestinal fluids (940 and 1390 μg/mL, respectively), fasted state simulated state gastric fluid (FaSSGF; pH 1.6, 1 h at 37° C.; 792 μg/mL) and aqueous PBS (pH 7.4, 1 h at 37° C.; 860 μg/mL). In both apical to basolateral (A-B) and basolateral to apical (B-A) directions across Caco-2 cell monolayers, Compound IE exhibited well-defined flux profiles with high mass balance allowing calculation of reliable A-B and B-A Pₐₚₚ values of 64 and 58×10⁻⁶ cm/s, respectively. The bidirectional Pₐₚₚ values indicate that the permeability for Compound IE is high and that this compound was not subject to apical efflux in the Caco-2 test system. Using the data for FaSSIF solubility and permeability, the solubility limited absorbable dose (SLAD, Butler et al, 2010) was estimated to be greater than 2 g.

As determined by rapid equilibrium dialysis, plasma protein binding for Compound IE in human and mouse plasma was 63.3±4.3 and 53.8±4.8%, respectively. The apparent human whole blood to plasma partitioning ratio for Compound IE was 0.78±0.035. Consistent with its high metabolic stability in human and mouse microsomes noted earlier, Compound IE was also metabolically stable in rat and dog microsomes with in vitro CLᵢₙₜ values<7 μL/min/mg protein. Similarly, Compound IE was minimally degraded in mouse, rat, dog, and human cryopreserved hepatocytes (in vitro CLᵢₙₜ values<2 mL/min/10⁶ cells). Compound IE at concentrations up to 20 μM did not inhibit the five major drug-metabolising cytochrome P450 (CYP) isoforms (CYPs 1A2, 2C9, 2C19, 2D6 and 3A4) in human liver microsomes.

Compound IC was evaluated in a similar fashion as described above for Compound IE. The results are summarized in Table 4.

TABLE 4
Summary of Properties of Compound IC
Physical Properties

| | |
|---|---|
| MW | 286.3 |
| Polar surface area | 49.4 Å |
| H-bond D/A | 1/2 |
| pKa (calc) | none |
| Log_{D7.4} (shake flask) | 2.1 |

Biorelevant Solubility

| | | Solubility |
|---|---|---|
| Media | pH | (μg/mL) |
| FaSSGF^{a} | 1.6 | 558 |
| FaSSIF (V2)^{b} | 6.5 | 1008 |
| FeSSIF (V2) b | 5.8 | 1205 |
| PBS^{b} | 7.4 | 836 |

| | CLᵢₙₜ | CLᵢₙₜ |
|---|---|---|
| Species | (μL/min/mg) | (μL/min/10⁶) |
| Human | | <1 |
| Dog | | <1.9 |
| Rat | | <1 |
| Mouse | | <1.8 |

Permeability

| | |
|---|---|
| Apical-basolateral | 70.4 × 10⁻⁶ |
| Basolateral-apical | 59.4 × 10⁻⁶ |

Protein binding, B:P

| matrix | % bound | B:P |
|---|---|---|
| human plasma | 66 | Not assessed |
| Dog plasma | 64 | Not assessed |
| Rat plasma | 62 | 1.2 |
| Mouse plasma | 62 | 0.95 |
| HLM | Not assessed | — |

CYP inhibition

| CYP | IC₅₀ (μm) |
|---|---|
| 1A2 | >20 |
| 2B6 | >20 |
| 2C8 | >20 |
| 2C9 | >20 |
| 2C19 | >20 |
| 2D6 | >20 |
| 3A4* (tes/mid) | >20 |

As shown in Tables 3 and 4, Compounds IE and IC had experimental shake-flask Log D_{7.4} value of 1.9 and 2.1, respectively. Moreover, compounds exhibited good solubility across a range of biorelevant media including fasted (FaSSIF-V2) and fed (FeSSIF-V2) state simulated intestinal fluids (>900 μg/mL), fasted state simulated gastric fluid (FaSSGF, 600-800 μg/mL) and aqueous PBS (pH 7.4, 850 μg/mL). Compounds IE and IC also exhibited high permeability across Caco-2 cell monolayers, bidirectional Papp values indicating that Compound IE and Compound IC are not subject to apical efflux. The compounds also exhibited moderate plasma protein binding (˜65%) across species for both compounds; high metabolic stability in mouse, rat, dog, and human microsomes (CLint<7 μL/min/mg protein); minimal degradation in mouse, rat, dog and human cryopreserved hepatocytes (CLint values<2 μL/min/106 cells); and no inhibition of the five major drug-metabolising cytochrome P450 (CYP) isoforms (CYPs 1A2, 2B6, 2C8, 2C9, 2C19, 2D6 and 3A4) at concentrations up to 20 μM in human liver microsomes.

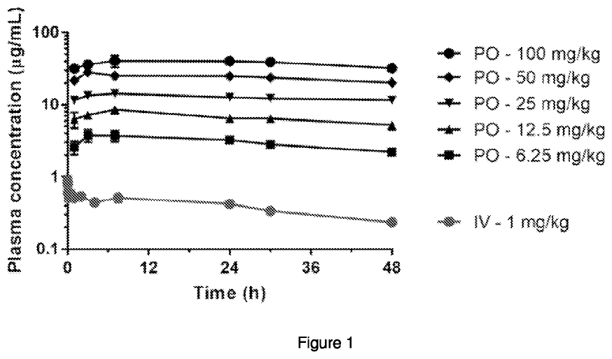

Table 5 summarizes the pharmacokinetic properties of Compound IE in mice upon intravenous and oral administration. The plasma concentration data for Compound IE in mice is shown in FIG. 1.

TABLE 5
Pharmacokinetics of Compound IE in mice

| Parameter | IV Administration |
|---|---|
| Mean Dose (mg/kg) | 1.0 |
| Apparent t_{1/2} (h) | 38 |
| Plasma CL (mL/min/kg) | 0.491 |
| Plasma_{VSS} (L/kg) | 1.6 |
| Plasma AUC_{0-48 h} (μg*h/mL) | 28.2 |
| Plasma AUC_{0-inf} (μg*h/mL) | 33.9 |

| Parameter | Oral Administration | | | | |
|---|---|---|---|---|---|
| Mean Dose (mg/kg) | 6.73 | 13.5 | 26.3 | 55.6 | 99.7 |
| Tₘₐₓ (h) | 3.0 | 7.0 | 7.0 | 3.0 | 7.0 |
| Plasma Cₘₐₓ (μg/mL) | 3.76 | 8.59 | 14.4 | 28.2 | 40.5 |
| Plasma Cₘₐₓ/Dose | 0.559 | 0.638 | 0.547 | 0.507 | 0.407 |
| (μg/mL)/(mg/kg) | | | | | |
| Plasma AUC_{0-48 h} | 145 | 320 | 607 | 1134 | 1799 |
| (μg*h/mL) | | | | | |
| Plasma AUC_{0-48 h}/Dose | 21.5 | 23.8 | 23.1 | 20.4 | 18.0 |
| (μg*h/mL)/(mg/kg) | | | | | |
| Apparent BA (%) | 76 | 84 | 82 | 72 | 64 |

As shown in FIG. 1, Compound IE exhibited high and prolonged exposure at doses of 6.25 to 100 mg/kg following oral administration to mice. The Cₘₐₓ and AUC values showed a generally proportional increase relative to the increase in dose (Table 5 and FIG. 1). The apparent oral bioavailability was high (at least about 80%, e.g., 95-100%) at all doses. Pharmacokinetic experiments of Compound IE in rats showed similar trends.

Compound IC, the non-deuterated analog of Compound IE, exhibited a high oral bioavailability in mice and rats, but a 2 to 4 times lower IV half-life and AUCs in mice (Table 6).

TABLE 6
Pharmacokinetics of Compound IC in mice

| Parameter | IV Administration |
|---|---|
| Mean Dose (mg/kg) | 1.0 |
| Apparent t_{1/2} (h) | 8.9 |
| Plasma CL (mL/min/kg) | 1.96 |
| Plasma_{VSS} (L/kg) | 1.64 |
| Plasma AUC_{0-48 h} (μg*h/mL) | 8.48 |
| Plasma AUC_{0-inf} (μg*h/mL) | 8.52 |

Using the data for FaSSIF solubility and permeability, the solubility limited absorbable dose (SLAD) was estimated to be greater than 2 g. This suggests that simple formulation approaches are likely to be suitable for Compound IE and Compound IC and that solubility-limited absorption is unlikely.

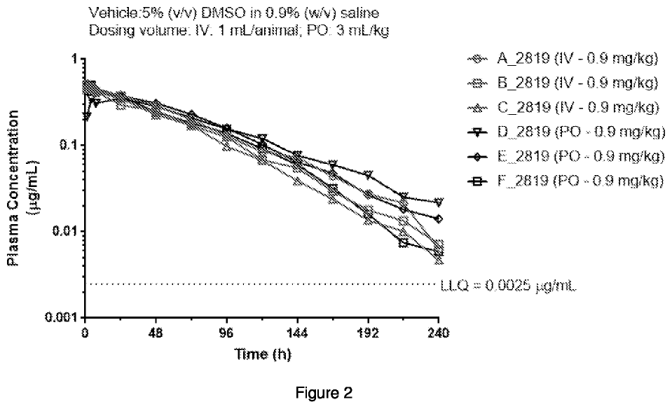

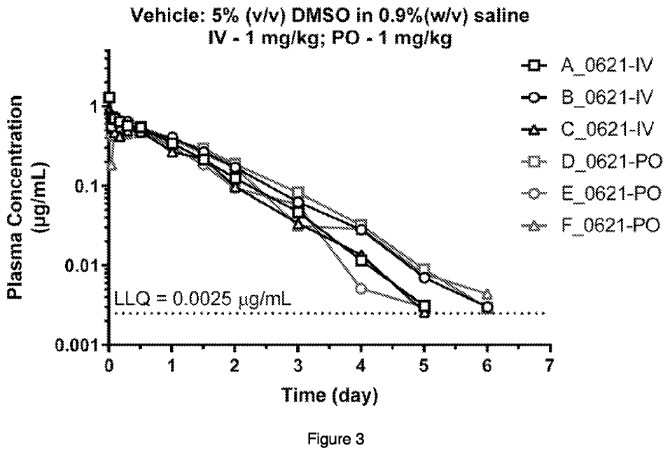

Compounds IE and IC were also studied in rats and the results are shown in Tables 7 and 8 and FIGS. 2 and 3.

TABLE 7
Pharmacokinetics of Compound IE in rats

| | | |
|---|---|---|
| | Mean | |
| Parameter | (n = 3) | SD |

IV administration

| | | |
|---|---|---|
| Dose (mg/kg) | 0.9 | — |
| Apparent t_{1/2} (h) | 32 | 2 |
| Plasma AUC_{IV} (h*ug/mL) | 31.7 | 3.3 |
| Plasma CL (mL/min/kg) | 0.476 | 0.048 |
| Plasma_{VSS} (L/kg) | 1.69 | 0.11 |

PO administration

| | | |
|---|---|---|
| Dose (mg/kg) | 0.9 | — |
| Apparent t_{1/2} (h) | 39 | 11 |
| Plasma AUCₒᵣₐₗ (H*ug/mL) | 36.0 | 2.8 |
| Plasma Cₘₐₓ (ug/mL) | 0.440 | 0.077 |
| Tₘₐₓ (h) | 11 | 12 |
| BA (%) | 113 | 9 |

TABLE 8
Pharmacokinetics of Compound IC in rats

| | | |
|---|---|---|
| Parameter | Mean (n = 3) | SD |

IV administration

| | | |
|---|---|---|
| Dose (mg/kg) | 1.0 | — |
| Apparent t_{1/2} (h) | 15 | 1.5 |
| Plasma AUC_{IV} (h*ug/mL) | 20.6 | 3.21 |
| Plasma CL (mL/min/kg) | 0.825 | 0.134 |
| Plasma_{Vss} (L/kg) | 1.25 | 0.16 |

PO administration

| | | |
|---|---|---|
| Dose (mg/kg) | 1.0 | — |
| Apparent t_{1/2} (h) | 16 | 3.0 |
| Plasma AUCₒᵣₐₗ (H*μg/mL) | 20.4 | 2.86 |
| Plasma Cₘₐₓ (μg/mL) | 0.572 | 0.037 |
| Tₘₐₓ (h) | 6.7 | 5.5 |
| BA (%) | 99.3 | 13.9 |

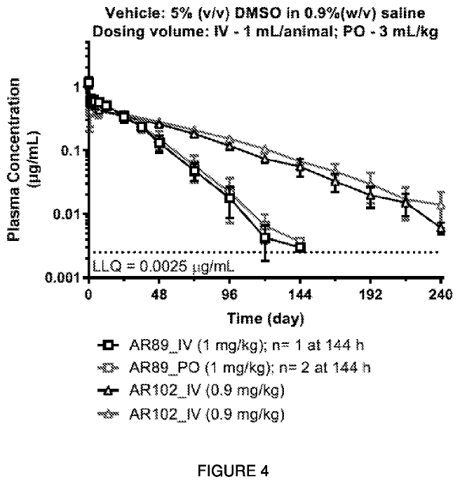

A comparison of plasma concentrations for Compounds IC and IE in male Sprague Dawley rats following IV and oral administration is shown in FIG. 4.

### Example 4. Toxicity Studies of Compound IE in Beagle Dogs

This example describes a pharmacokinetic study of Compound IE in male and female Beagle dogs.

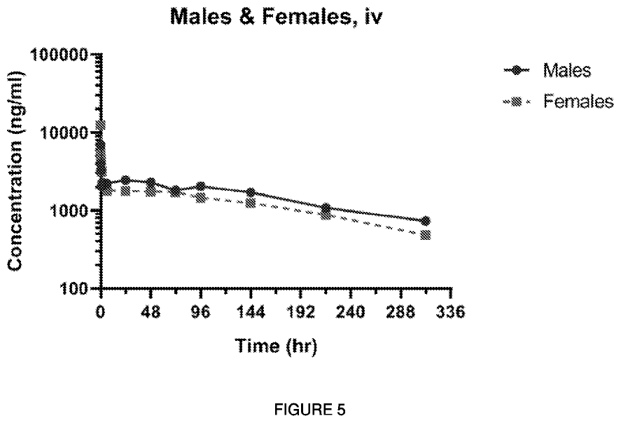

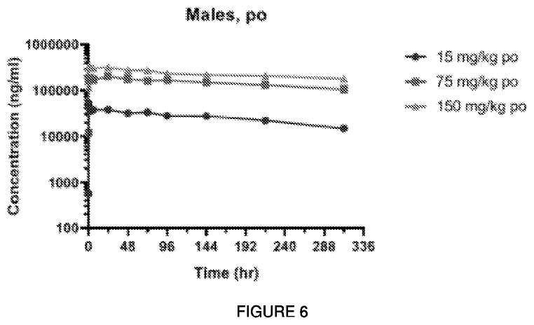

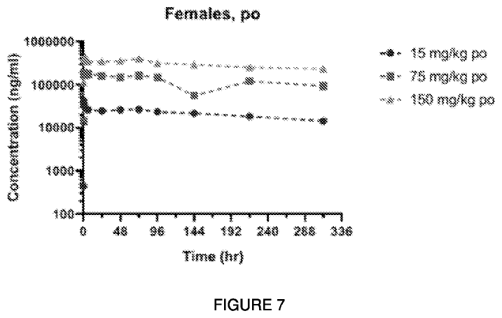

The objectives of this study were to identify potential dose limiting toxicities and determine pharmacokinetic (PK) properties of Compound IE in beagle dogs following a single oral (po) or intravenous (iv) dose administration. The experimental design is summarized in Table 15 and plasma concentration data are shown in FIGS. 5-8.

TABLE 9
Experimental Design

| | | | | | Total | |
|---|---|---|---|---|---|---|
| | Dose | Dose | Dose Conc. | Dose Vol | No. of | Blood Collection |
| Group | Route | (mg/kg) ^{a} | (mg/ml) | (ml/kg) ^{b} | Animals | Timepoints |
| 1 | iv | 1 | 0.4 | 2.5 | 2M/2F | Predose, 5, 15 min, 0.5, |
| | | | | | | 1, 2, 6, 24, 48, 72, 96 |
| | | | | | | hr; and 7, 10 and 14 days |
| 2 | po | 15 | 3 | 5 | 2M/2F | Predose, 15 min, 0.5, 1, |
| | | | | | | 2, 6, 24, 48, 72, 96 hr; |
| | | | | | | and 7, 10 and 14 days |
| 3 | po | 75 | 15 | 5 | 2M/2F | Predose, 15 min, 0.5, 1, |
| | | | | | | 2, 6, 24, 48, 72, 96 hr; |
| | | | | | | and 7, 10 and 14 days |
| 4 | po | 150 | 30 | 5 | 2M/2F | Predose, 15 min, 0.5, 1, |
| | | | | | | 2, 6, 24, 48, 72, 96 hr; |
| | | | | | | and 7, 10 and 14 days |

^{a} Test article was administered as a single iv or po dose on Day 1. The same 2M/2F were used after 2-week washout periods for each dose level in Group number order.
^{b} The dose volumes were based on each animal's Day 1 body weight.

Male and female Beagle dogs (2/sex/group) were given a single administration of Compound IE either iv at 1 mg/kg or po at 15, 75 and 150 mg/kg. The same 2M/2F dogs were used following 2-week washout periods for each dose level in escalation order. This was a survival study and animals were not sacrificed. The following parameters were evaluated: mortality/morbidity, clinical observations, body weights, plasma drug levels, pharmacokinetics and clinical pathology (hematology, coagulation and serum chemistry).

No early deaths or moribund animals were observed following the single iv or po treatments. No relevant test article-related effects in clinical observations were observed after dose administration; however, hypoactivity, emesis, ataxia, lacrimation and swelling from both eyes, skin discolored pink on both ears and “wet dog” shakes on Day 1, immediately after the iv dose, were observed. These observations are consistent with a moderate acute hypersensitivity reaction believed to be triggered by the vehicle component Tween 80 which has been implicated in severe hypersensitivity reactions in humans and dogs (Qiu et al., 2013; Boccia et al., 2019). Other findings of soft stool, diarrhea, emesis, slight hypoactivity and drooling were observed after po dose. Many of these findings are commonly observed in Beagle dogs and are not considered to be toxicologically relevant. There were no meaningful drug-related effects seen in body weight or clinical pathology parameters.

Pharmacokinetic parameters for the plasma concentrations of Compound IE were determined. The lower limit of quantitation (LLOQ) for the assay was 100 ng/mL. After iv administration of 1 mg/kg Compound IE, the mean peak plasma concentrations (Cₘₐₓ) were 6.88 μg/mL (males) and 12.5 μg/mL (females) and the mean plasma concentration values extrapolated to time zero (C₀) were 9.46 μg/ml and 18.8 μg/ml for male and female dogs, respectively. The mean area under the plasma concentration curve to the last timepoint (AUCₗₐₛₜ) values after the iv dose were 485 h˜μg/mL (males) and 377 h·μg/mL (females). The mean elimination phase half-life (t_{1/2}) values were 167 h and 152 h for male and female dogs, respectively. See FIG. 5.

The time of peak plasma concentrations (Tₘₐₓ) was highly variable in the po dose groups, ranging from 0.25 hr (15 mg/kg both males and females) to 24 hr (male 9, 75 mg/kg and male 13, 150 mg/kg). Exposure to Compound IE based on the AUClast values increased approximately in proportion to dose, e.g., a 5- to 6-fold increase for 75 mg/kg dose relative to 15 mg/kg, and a 2- to 3-fold increase for the 150 mg/kg compared to mid-dose. The mean t₁/2 values for Compound IE after oral administration for males varied from 237 hr (15 mg/kg) to 341 hr (75 mg/kg) for male dogs, and from 48 hr (15 mg/kg) to 402 hr (150 mg/kg) for females. The area under the curve to infinity (AUC_{inf}) values could not be accurately calculated because 20% to 75% of the curve was extrapolated due to the extended period of time that Compound IE remained in the systemic circulation. Bioavailability, determined using AUCₗₐₛₜ values, was greater than 100% in most animals which may be due to slow absorption from the GI tract resulting in sustained release exposure.

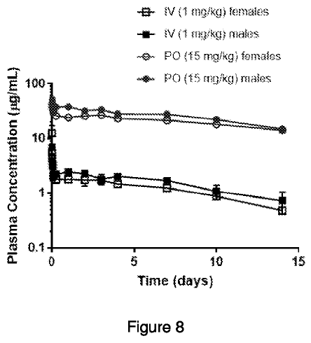

A comparison of plasma concentrations for Compound IE in female and male dogs after iv administration (1 mg/kg) or po (15 mg/kg) is shown in FIG. 8 and summarized in Table 10.

TABLE 10
Pharmacokinetics of Compound IE in dogs

| | | |
|---|---|---|
| | Females | Males |
| Parameter | (n = 2) | (n = 2) |

IV administration

| | | |
|---|---|---|
| Dose (mg/kg) | 1 | 1 |
| Apparent t_{1/2} (h) | 123 | 155 |
| Plasma AUC_{IV 0-last} (h*ug/mL) | 405 | 526 |
| Plasma CL (mL/min/kg) | 0.032 | 0.024 |
| Plasma_{Vss} (L/kg) | 0.45 | 0.36 |

PO administration

| | | |
|---|---|---|
| Dose (mg/kg) | 15 | 15 |
| Apparent t_{1/2} (h) | 348 | 237 |
| Plasma AUC_{oral 0-last} (h*ug/mL) | 7033 | 8795 |
| Plasma _{Cmax} (μg/mL) | 42.7 | 51.9 |
| Tₘₐₓ | 0.25 | 0.25 |
| BA (%) based on AUC₀₋ₗₐₛₜ | 115 | 111 |

In conclusion, a single administration of Compound IE, given either iv at 1 mg/kg or po at 15, 75 and 150 mg/kg to male and female Beagle dogs produced no overt toxicity. In the absence of any Compound IE-related effects on clinical observations, body weight or clinical pathology parameters, the maximum tolerated dose (MTD) could not be established but is considered to be greater than 1 mg/kg as a single iv administration or 150 mg/kg as a single po administration. The no observed adverse effect level (NOAEL) of Compound IE in both male and female Beagle dogs is considered to be at least 1 mg/kg as a single iv administration or 150 mg/kg as a single po administration.

### Example 5—Pharmacokinetic Studies of Compound IC in Rats

The pharmacokinetics of Compound IC were evaluated in male Sprague Dawley rats as described herein using the conditions shown in Table 11.

TABLE 11

| Dose route | IV | | PO | |
|---|---|---|---|---|
| Target Dose | 1 | mg/kg | 1 | mg/kg |
| Dosing details | 10 min infusion via an indwelling catheter into | | Via an oral gavage - 3 | |
| | the jugular vein - 1 mL dose volume per | | mL/kg dose volume | |
| | animal | | | |
| Post-dose plasma | During infusion: 10 min; post- dose: 1, 2, 4, 7, | | 1, 2, 4, 7, 12, 24, 36, 48, | |
| collection^{a} | 12, 24, 36, 48, 72, 96, 120, 144 and 168 h | | 72, 96, 120,144 and 168 h | |
| Vehicle | 5% (v/v) DMSO in 0.9% (w/v) saline | | | |
| Appearance | Solution with an apparent pH of 5.0 | | Solution with an apparent | |
| | | | pH of 5.2 | |
| Target | 0.25 | mg/mL | 0.33 | mg/mL |

Concentration

^{a}plasma samples were taken from each rat prior to dosing for use as analytical blanks

There were no adverse reactions or compound-related side effects observed in any rats during the 7-day sampling period after IV or oral administration of Compound IC. The apparent whole blood-to-plasma ratio (B/P) of Compound IC in rat blood was 1.2.

The plasma concentration versus time profiles of Compound IC after IV and oral administration to male Sprague Dawley rats are shown in FIG. 3 while the pharmacokinetic parameters are presented in Tables 8, 12, and 14.

A comparison of the Compound IC profiles with data for Compound IE is shown in FIG. 4. Following IV and oral administration of Compound IC, measurable plasma concentrations were observed for up to 6 days, and profiles exhibited an apparent half-life of approximately 13-19 h, which is much shorter than that for Compound IE (32±2 h, FIG. 2). The apparent blood volume of distribution for Compound IC was moderate and blood clearance was very low. Assuming that Compound IC was eliminated primarily by hepatic metabolism, the ratio of the blood clearance to the nominal hepatic blood flow in the rat (67.6 mL/min/kg) suggests that Compound IC has an extremely low in vivo hepatic extraction ratio (approximately 0.01).

Following oral administration, plasma concentrations were either at or close to Cₘₐₓ at 1-2 h and the apparent bioavailability was approximately 100%. Quantitative absorption (at this relatively low dose) is consistent with the high aqueous solubility of Compound IC (>100 μg/mL at pH 6.5) and other physicochemical properties that are within the ranges typically associated with good membrane permeability.

TABLE 12
Pharmacokinetic parameters for Compound IC in male Sprague Dawley rats following
IV and oral administration.
IV administration at 1 mg/kg

| Parameter | A_0621 | B_0621 | C_0621 | Mean | SD |
|---|---|---|---|---|---|
| Apparent t_{1/2} (h) | 13 | 16 | 14 | 15 | 1.5 |
| Plasma AUC_{IV} (h*μg/mL) | 20.9 | 23.6 | 17.2 | 20.6 | 3.21 |
| Plasma CL (mL/min/kg) | 0.797 | 0.707 | 0.970 | 0.825 | 0.134 |
| Plasma Vₛₛ (L/kg) | 1.14 | 1.19 | 1.43 | 1.25 | 0.156 |
| B/P ratio | 1.2 | | | | |
| Blood CL (mL/min/kg) | 0.664 | 0.589 | 0.808 | 0.687 | 0.111 |
| Blood Vₛₛ (L/kg) | 0.948 | 0.991 | 1.19 | 1.04 | 0.130 |

PO administration at 1 mg/kg

| Parameter | D_0621 | E_0621 | F_0621 | Mean | SD |
|---|---|---|---|---|---|
| Apparent t_{1/2} (h) | 16 | 13 | 19 | 16 | 3.0 |
| Plasma AUCₒᵣₐₗ (h*μg/mL) | 23.3 | 17.6 | 20.4 | 20.4 | 2.86 |
| Plasma Cₘₐₓ (μg/mL) | 0.614 | 0.559 | 0.543 | 0.572 | 0.0368 |
| Tₘₐₓ (h) | 1.0 | 7.0 | 12 | 6.7 | 5.5 |
| BA (%) | 113 | 85.5 | 99.2 | 99.3 | 13.9 |

IV and oral formulations were prepared using the same method. On the day of dosing the solid compound was dissolved in DMSO prior to addition of a saline solution (0.9% w/v). The samples were then mixed using vortexing producing colourless solutions. The IV formulation was filtered through a 0.22 μm syringe filter prior to dosing and the oral formulation was dosed without filtration.

The concentration of Compound IC in each formulation was determined via a suitably validated generic HPLC-UV assay using a Waters Acquity HPLC system with a Phenomenex Ascentis Express RP-Amide column (50×2.1 mm, 2.7 μm) coupled to a Waters PDA detector analysing at 254 nm. The measured concentrations of Compound IC in aliquots of the IV and oral formulations are presented in Table 11.

All animal studies were conducted using established procedures in accordance with the Australian Code of Practice for the Care and Use of Animals for Scientific Purposes, and the study protocols were reviewed and approved by the Monash Institute of Pharmaceutical Sciences Animal Ethics Committee.

The pharmacokinetics of Compound IC were studied in overnight-fasted male Sprague Dawley rats that had access to water ad libitum throughout the pre- and post-dose sampling period, and access to food was re-instated 4 h post-dose. In-life details are provided in Table 13.

Compound IC was administered intravenously as a 10 min constant rate infusion via an indwelling jugular vein cannula (1 mL per rat, n=3 rats) and orally by gavage (3 mL/kg per rat, n=3 rats). Samples of arterial blood were collected up to 168 h post-dose. Urine samples were not collected as animals were housed in bedded cages. Arterial blood was collected directly into borosilicate vials (at 4° C.) containing heparin as anticoagulant. Once collected, blood samples were centrifuged, supernatant plasma was removed and stored frozen (−80° C.) until analysis by LC-MS. A summary of the bioanalytical method and assay validation details is included in Tables 14 and 15.

TABLE 13
In-life summary

| Species (strain) | Rat (Sprague Dawley) | |
|---|---|---|
| Sex (age) | Male (7-8 weeks) | |
| Dose route | IV | Oral gavage |

| | | | | |
|---|---|---|---|---|
| Nominal dose level | 1 | mg/kg | 1 | mg/kg |
| Dose volume | 1 | mL per rat | 3 | mL/kg |
| Measured formulation | 0.23 | mg/mL | 0.31 | mg/mL |
| concentration | | | | |

| Rat ID | A_0621 | B_0621 | C_0621 | D_0621 | E_0621 | F_0621 |
|---|---|---|---|---|---|---|
| Weight (g) | 250 | 251 | 245 | 263 | 268 | 258 |
| Dose volume (mL) | 1.0 | 1.0 | 1.0 | 0.79 | 0.80 | 0.77 |

### In Vivo Determination of Whole Blood-to-Plasma Ratio

As part of the assessment of the IV pharmacokinetics, an additional blood sample was collected from each rat at 1 h post dose for the purposes of determining the whole blood-to-plasma (B/P) ratio. Arterial blood was collected into tubes containing heparin. The haematocrit was determined via centrifugation (13000×g for 4 min using a Clemets® Microhematocrit centrifuge and Safecap® Plain Self-sealing Mylar Wrapped capillary tubes), and values ranged from 41% to 42%. Each blood aliquot (25 pL) was collected and matrix matched with an equivalent volume of blank plasma. The remainder of the blood sample was centrifuged and 1×25 pL plasma aliquot was collected and matrix matched with an equivalent volume of blank blood. Samples were frozen on dry ice and stored at −80° C. until analysis by LC-MS.

The B/P ratio was calculated on the basis of the LC-MS response of Compound IC in blood and plasma following centrifugation of whole blood and the average B/P ratio (n=3 rats) is reported.

### Bioanalytical Method Summary

Concentrations of test compound in plasma samples were determined using an LC-MS/MS method validated for linearity, accuracy, precision, matrix factor and recovery (Table 14).

Test compound standard solutions were diluted from a concentrated stock solution (1 mg/mL in DMSO) using 50% acetonitrile in water (v/v) and a calibration curve was prepared in a matched matrix to the test samples.

Plasma: The plasma calibration curve was prepared by spiking aliquots of blank rat plasma (50 pL) with test compound standard solutions (10 pL) and internal standard solution (10 pL of diazepam, 5 μg/mL in 50% acetonitrile in water). Test plasma samples (50 pL) were thawed, mixed and then spiked with internal standard solution (10 pL). Plasma protein precipitation was performed by addition of acetonitrile (3-fold volume ratio) and thorough vortex mixing. Samples were centrifuged (RCF=9391×g) for 3 minutes and the supernatant was collected for analysis. The analysis of blood/plasma (B/P) partitioning samples was conducted similarly as for the plasma samples, using protein precipitation with acetonitrile as described above.

Replicate analysis: Triplicate analytical replicate (ARs) samples were prepared similarly to the standards for each sample type at three concentrations (50, 500 and 2000 ng/mL) and repeat injections of these ARs were included throughout the analytical run to assess assay performance. The extraction of the test compound from the standards, and ARs were conducted as described above.

All test samples were quantified within the calibration range of the assay and the assay performance for ARs were acceptable (Table 15). The plasma matrix factor was 83% and the recovery of the test compound from rat plasma was 89%.

TABLE 14
Analytical Replicates (AR) and calibration data

| | |
|---|---|
| Instrument | Waters Xevo TQD coupled to a Waters Acquity UPLC |
| Detection | Negative electrospray ionisation multiple-reaction monitoring mode |
| Column | Supelco Ascentis Express RP C8 column (50 × 2.1 mm, 2.7 pm) |
| LC conditions | Gradient cycle time: 4 min; Injection vol: 6 pL; Flow rate: 0.4 mL/min |
| Mobile phase | (A) 0.05% formic acid in water; (B) 0.05% formic acid in acetonitrile |
| Sample Preparation | Protein precipitation using acetonitrile (1:3 volume ratio) |

| Analyte | tR* (min) | Transition (m/z) | Cone voltage (V) | CID^{#} (V) |
|---|---|---|---|---|
| Compound IC | 2.2 | 284.99 > 174.19 | 45 | 20 |
| Diazepam (IS) | 2.4 | 285.12 > 154.09 | 50 | 25 |

The highest abundance product ion with minimum interference with the matrix were selected for quantification. Data acquisition was performed using MassLynx software (V4.2).
IS: Internal standard
*Retention time
^{#}Collision-Induced Dissociation

TABLE 15
Analytical Replicates (AR) and calibration data

| | Accuracy and Precision\hat{ } | | | | | | | |
|---|---|---|---|---|---|---|---|---|
| | | | | Accuracy | Precision | Calibration Data^{~} | | |
| Matrix | Sample | Conc. | | (% bias) | (% RSD) | Range | R² | LLQ |
| Plasma | ARs | 50 | (n = 9) | 9.1 | 7.9 | 2.5-10000 | 0.9997 | 2.5 |
| (ng/mL) | | 500 | (n = 9) | 5.4 | 3.8 | | | |
| | | 2000 | (n = 9) | 2.9 | 2.8 | | | |

Acceptance criteria for batch analysis: at least 67% of the AR samples must be within ±15% of nominal values (CDCO In-house acceptance criteria).
^{~}Calibration data were fitted to quadratic equation with a weighting factor of 1/x. The lower limit of quantitation (LLQ) was defined by the lowest acceptable calibration standard for which the back calculated concentration was within ±20% of the nominal concentration.

### Standard Calculations

The measured concentrations in both the IV and the oral formulations were within 15% of the nominal concentrations, hence the nominal doses were used for data analysis. Plasma concentration versus time data were analysed using non-compartmental methods (PKSolver Version 2.0). Standard calculations for each pharmacokinetic parameter are listed below.

$$Plasma CL = \frac{{Dose}_{IV}}{{AUC}_{IV}}$$

$$Plasma {V}_{ss} = \frac{{AUMC}_{IV}}{{AUC}_{IV}} \times Plasma CL$$

$${t}_{1 / 2} = \frac{Ln ( 2 )}{{λ}_{z}}$$

$$Blood CL = \frac{Plasma CL}{B / P}$$

$$Blood {V}_{ss} = \frac{Plasma {V}_{ss}}{B / P}$$

$$BA = \frac{{AUC}_{oral} \times {Dose}_{IV}}{{AUC}_{IV} \times {Dose}_{oral}} \times 100 \%$$

CL Clearance in plasma/blood after IV administration
AUC_{IV} Area under the plasma concentration versus time profile from time zero to infinity after IV administration
B/P Whole blood-to-plasma partitioning ratio
t_{1/2} Elimination half-life
λ_{z} Terminal elimination rate constant after IV/oral administration
Vₛₛ Apparent volume of distribution in plasma/blood at steady state
AUMC_{IV} Area under the first moment of the plasma concentration versus time profile from time zero to infinity after V administration
BA Oral bioavailability
AUCₒᵣₐₗ Area under the plasma concentration versus time profile from time zero to infinity after oral administration
Cₘₐₓ Maximum plasma concentration observed after oral administration
Tₘₐₓ Time to achieve Cₘₐₓ

### Standard Calculations

The measured concentrations in both the IV and the oral formulations were within 15% of the nominal concentrations, hence the nominal doses were used for data analysis. Plasma concentration versus time data were analysed using non-compartmental methods (PKSolver Version 2.0). Standard calculations for each pharmacokinetic parameter are listed below.

$$Plasma CL = \frac{{Dose}_{IV}}{{AUC}_{IV}}$$

$$Plasma {V}_{ss} = \frac{{AUMC}_{IV}}{{AUC}_{IV}} \times Plasma CL$$

$${t}_{1 / 2} = \frac{Ln ( 2 )}{{λ}_{z}}$$

$$Blood CL = \frac{Plasma CL}{B / P}$$

$$Blood {V}_{ss} = \frac{Plasma {V}_{ss}}{B / P}$$

$$BA = \frac{{AUC}_{oral} \times {Dose}_{IV}}{{AUC}_{IV} \times {Dose}_{oral}} \times 100 \%$$

CL Clearance in plasma/blood after IV administration
AUC_{IV} Area under the plasma concentration versus time profile from time zero to infinity after IV administration
B/P Whole blood-to-plasma partitioning ratio
t_{1/2} Elimination half-life
λ_{z} Terminal elimination rate constant after IV/oral administration
Vₛₛ Apparent volume of distribution in plasma/blood at steady state
AUMC_{IV} Area under the first moment of the plasma concentration versus time profile from time zero to infinity after IV administration
BA Oral bioavailability
AUCₒᵣₐₗ Area under the plasma concentration versus time profile from time zero to infinity after oral administration
Cₘₐₓ Maximum plasma concentration observed after oral administration
Tₘₐₓ Time to achieve Cₘₐₓ

TABLE 16
Plasma concentrations of Compound IC in male Sprague
Dawley rats following IV and oral administration
IV Administration of 1 mg/kg

| | Plasma Concentration (ng/mL) | | | | |
|---|---|---|---|---|---|
| Time (h) | A_0621 | B_0621 | C_0621 | Mean | SD |
| 0.17 | 1292 | 1300 | 908 | 1167 | 224 |
| 1.17 | 728 | 549 | 529 | 602 | 110 |
| 2.17 | 701 | 669 | 469 | 613 | 126 |
| 4.17 | 640 | 662 | 420 | 574 | 134 |
| 7.17 | 569 | 641 | 493 | 568 | 73.7 |
| 12.17 | 545 | 473 | 480 | 499 | 39.4 |
| 24.17 | 340 | 403 | 268 | 337 | 67.7 |
| 36.17 | 215 | 266 | 216 | 232 | 29.5 |
| 48.17 | 125 | 170 | 97 | 130 | 36.8 |
| 72.17 | 46.8 | 62.2 | 33.5 | 47.5 | 14.4 |
| 96.17 | 11.5 | 28.1 | 13.6 | 17.7 | 9.0 |
| 120.17 | 3.1 | 7.0 | 2.6 | 4.2 | 2.4 |
| 144.17 | ND | 3.0 | ND | 3.0 | — |
| 168.17 | ND | ND | ND | — | — |

Oral Administration of 1 mg/kg

| | Plasma Concentration (ng/mL) | | | | |
|---|---|---|---|---|---|
| Time (h) | D_0621 | E_0621 | F_0621 | Mean | SD |
| 1.00 | 614 | 430 | 184 | 409 | 216 |
| 2.00 | 612 | 506 | 427 | 515 | 92.9 |
| 4.00 | 545 | 477 | 474 | 499 | 40.3 |
| 7.00 | 612 | 559 | 455 | 542 | 79.8 |
| 12.00 | 475 | 499 | 543 | 506 | 34.8 |
| 24.00 | 365 | 309 | 390 | 355 | 41.3 |
| 36.00 | 294 | 183 | 254 | 243 | 56.1 |
| 48.00 | 188 | 94 | 170 | 151 | 49.9 |
| 72.00 | 82.4 | 58.1 | 31.5 | 57 | 25.4 |
| 96.00 | 32.0 | 5.1 | 28.5 | 21.8 | 14.6 |
| 120.00 | 9.0 | 3.0 | 7.8 | 6.6 | 3.2 |
| 144.00 | 2.9 | ND | 4.4 | 3.7 | 1.0 |
| 168.00 | ND | ND | ND | — | — |

ND Not detected

### REFERENCES

Bergquist, R.; Utzinger, J.; Keiser, J. Controlling schistosomiasis with praziquantel: How much longer without a viable alternative? Infect. Dis. Poverty 2017, 6, 74.
Bernauer, K.; Link, H.; Stohler, H. Antiandrogenic and schistosomicidal imidazolidine derivatives. U.S. Pat. No. 4,234,736. 1980.
Boccia RI, Geller RB, Clendeninn N and Ottoboni T. Hypersensitivity and infusion-site adverse events with intravenous fosaprepitant after anthracycline-containing chemotherapy: a retrospective study. Future Oncol. 15(3) (2019):297-303. doi: 10.2217/fon-2018-0662. Epub 2018 Oct. 10.
Butler, J. M.; Dressman, J. B. The developability classification system: Application of biopharmaceutics concepts to formulation development. J. Pharm. Sci. 2010, 99, 4940-4954.
Caffrey, C. R.; El-Sakkary, N.; MAder, P.; Krieg, R.; Becker, K.; Schlitzer, M.; Drewry, D. H.; Vennerstrom, J. L.; Grevelding, C. G. Drug discovery and development for schistosomiasis in Neglected Tropical Diseases: Drug Discovery and Development, Swinney, D.; Pollastri, M., Ed.'s, Wiley-VCH, Weinheim, Germany 2019. pp. 187-225.
Caffrey, C. R.; Secor, W. E. Schistosomiasis: From drug deployment to drug development. Curr. Opin. Infect. Dis. 2011, 24, 410-417.
Colley, D. G.; Bustinduy, A. L.; Secor, W. E.; King, C. H. Human schistosomiasis. Lancet 2014, 383, 2253-2264.
Gangwar, R. S.; Friedman, S.; Seaf, M.; Levi-Schaffer, F. Mast cells and eosinophils in allergy: Close friends or just neighbors. Eur. J. Pharmacol. 2016, 778, 77-83.
Geary, T. G.; Woo, K.; McCarthy, J. S.; Mackenzie, C. D.; Horton, J.; Prichard, R. K.; de Silva, N. R.; Olliaro, P. L.; Lazdins-Helds, J. K.; Engels, D. A.; Bundy, D. A. Unresolved issues in anthelmintic pharmacology for helminthiases of humans. Int. J. Parasitol. 2010, 40, 1-13.
Gryseels, B. Schistosomiasis. Infect. Dis. Clin. North Am. 2012, 26, 383-397.
Horton, J. Human gastrointestinal helminth infections: Are they now neglected diseases?Trends Parasitol. 2003, 19, 527-531.
Hotez, P. J.; Brindley, P. J.; Bethony, J. M.; King, C. H.; Pearce, E. J.; Jacobson, J. Helminth infections: The great neglected tropical diseases. J. Clin. Invest. 2008, 118, 1311-1321.
Jones, J. O.; Diamond, M. I. A cellular conformation-based screen for androgen receptor inhibitors. ACS Chem. Biol. 2008, 3, 412-418.
Kasinathan, R. S.; Greenberg, R. M. Pharmacology and potential physiological significance of schistosome multidrug resistance transporters. Exp. Parasitol. 2012, 132, 2-6.
Keiser, J.; Manneck, T.; Vargas, M. Interactions of mefloquine with praziquantel in the Schistosoma mansoni mouse model and in vitro. J. Antimicrob. Chemother. 2011, 66, 1791-1797.
Keiser, J.; Utzinger, J. Antimalarials in the treatment of schistosomiasis. Curr. Pharm. Des. 2012, 18, 3531-3538.
Keiser, J.; Vargas, M.; Vennerstrom, J. L. Activity of antiandrogens against juvenile and adult Schistosoma mansoni in mice. J. Antimicrob. Chemother. 2010, 65, 1991-1995.
Keiser, J.; Panic, G.; Vargas, M.; Wang, C.; Dong, Y.; Gautam, N.; Vennerstrom, J. L. Aryl hydantoin Ro 13-3978, a broad spectrum antischistosomal. J. Antimicrob. Chemother. 2015, 70, 1788-1797.
Knopp, S.; Becker, S. L.; Ingram, K. J.; Keiser, J.; Utzinger, J. Diagnosis and treatment of schistosomiasis in children in the era of intensified control. Expert Rev. Antiinfect. Ther. 2013, 11, 1237-1258.
Link, H.; Stohler, H. R. 3-Arylhydantoine, eine substanzklasse mit schistosomizider wirkung. Eur. J. Med. Chem.-Chim. Ther. 1984, 19, 261-265.
Mäder, P.; Rennar, G. A.; Ventura, A. M. P.; Grevelding, C. G.; Schlitzer, M. Chemotherapy for fighting schistosomiasis: Past, present and future. ChemMedChem 2018, 13, 2374-2389.
Melman, S. D.; Steinauer, M. L.; Cunningham, C.; Kubatko, L. S.; Mwangi, I. N.; Wynn, N. B.; Mutuku, M. W.; Karanja, D. M.; Colley, D. G.; Black, C. L.; Secor, W. E.; Mkoji, G. M.; Loker, E. S. Reduced susceptibility to praziquantel among naturally occurring Kenyan isolates of Schistosoma mansoni. PLoS Negl. Trop. Dis. 2009, 3, e504.
Murray, C. J. L.; GBD 2017 DALYs and HALE Collaborators. Global, regional, and national disability-adjusted life-years (DALYs) for 359 diseases and injuries and healthy life expectancy (HALE) for 195 countries and territories, 1990-2017: a systematic analysis for the Global Burden of Disease Study 2017. Lancet 2018, 392, 1859-1922.
Njoroge, M.; Njuguna, N. M.; Mutai, P.; Ongarora, D. S.; Smith, P. W.; Chibale, K. Recent approaches to chemical discovery and development against malaria and the neglected tropical diseases human African trypanosomiasis and schistosomiasis. Chem. Rev. 2014, 114, 11138-11163.
Olliaro, P.; Delgado-Romero, P.; Keiser, J. The little we know about the pharmacokinetics and pharmacodynamics of praziquantel (racemate and R-enantiomer). J. Antimicrob. Chemother. 2014, 69, 863-870.
Panic, G.; Keiser, J. Acting beyond 2020: Better characterization of praziquantel and promising antischistosomal leads. Curr. Opin. Pharmacol. 2018, 42, 27-33.
Panic, G.; Ruf, M.-T.; Keiser, J. Immunohistochemical Investigations of treatment with Ro 13-3978, praziquantel, oxamniquine, and mefloquine in Schistosoma mansoni-infected mice. Antimicrob. Agents Chemother. 2017, 61, e01142-17.
Pedrique, B.; Strub-Wourgaft, N.; Some, C.; Olliaro, P.; Trouiller, P.; Ford, N.; Pecoul, B.; Bradol, J.-H. The drug and vaccine landscape for neglected diseases (2000-11): A systematic assessment. Lancet Glob. Health 2013, 1, e371-e379.
Qiu S, Liu Z, Hou L, Li Y, Wang J, Wang H, Du W, Wang W, Qin Y and Liu Z. Complement activation associated with polysorbate 80 in beagle dogs. International Immunopharmacology, 15 (2013) 144-149.
Steinmann, P.; Keiser, J.; Bos, R.; Utzinger, J. Schistosomiasis and water resources development: Systematic review, meta-analysis, and estimates of people at risk. Lancet Infect. Dis. 2006, 6, 411-425.
Terer, C. C.; Bustinduy, A. L.; Magtanong, R. V.; Muhoho, N.; Mungai, P. L.; Muchiri, E. M.; Kitron, U.; King, C. H.; Mutuku, F. M. Evaluation of the health-related quality of life of children in Schistosoma haematobium-endemic communities in Kenya: A cross-sectional study. PLoS Negl. Trop. Dis. 2013, 7, e2106.
Utzinger, J.; N'goran, E. K.; Caffrey, C. R.; Keiser, J. From innovation to application: Social-ecological context, diagnostics, drugs and integrated control of schistosomiasis. Acta Trop. 2011, 120, S121-S137.
Vandyck, K.; Hache, G. Y. P; Last, S. J.; Mc Gowan, D. C.; Rombouts, G.; Verschueren, W. G.; Jean-Marie Bernard Raboisson, P. Sulphamoylpyrrolamide derivatives and the use thereof as medicaments for the treatment of hepatitis B. U.S. Patent WO2014/184350AI, 20 Nov. 2014.
Wang, C.; Zhao, Q.; Vargas, M.; Jones, J. O.; White, K. L.; Shackleford, D. M.; Chen, G.; Saunders, J.; Ng, A. C. F.; Chiu, F. C. K.; Dong, Y.; Charman, S. A.; Keiser, J.; Vennerstrom, J. L. Revisiting the SAR of the antischistosomal aryl hydantoin (Ro 13-3978). J. Med. Chem. 2016, 59,10705-10718.

What is claimed:

1. A compound, or pharmaceutically acceptable salt thereof, having the structure of Formula (I): 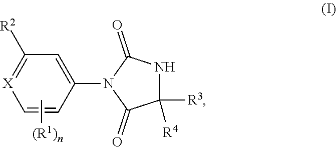
wherein
X is CH, CF, or N;
each R¹ is F;
n is 0-3;
R² is selected from F, C, Br, CF₂H, CF₃, CFH₂, CF₂CH₃, CFHCFH₂, CH₂CF₃, and OCF₃;
each of R³ and R⁴ is independently selected from CH₃, CD₃, CFH₂, CF₂H, CF₃, CF₂CH₃, CFHCFH₂, CH₂CF₃, and OCF₃; and
when X is CF, R² is CF₃, and n is 0, R³ and R⁴ are not both CH₃.

2. The compound or pharmaceutically acceptable salt of claim 1, wherein X is CF.

3. The compound or pharmaceutically acceptable salt of claim 1, wherein n is 0.

4. The compound or pharmaceutically acceptable salt of claim 1, wherein R² is CF₃.

5. The compound or pharmaceutically acceptable salt of claim 1, wherein each of R³ and R⁴ is independently selected from CH₃, CF₃ or CF₂H, and CD₃.

6. The compound or pharmaceutically acceptable salt of claim 5, wherein at least one of R³ or R⁴ is CH₃.

7. The compound or pharmaceutically acceptable salt of claim 5, wherein at least one of R³ or R⁴ is CF₃.

8. The compound or pharmaceutically acceptable salt of claim 5, wherein at least one of R³ or R⁴ is CF₂H.

9. The compound or pharmaceutically acceptable salt of claim 5, wherein at least one of R³ or R⁴ is CD₃.

10. The compound or pharmaceutically acceptable salt of claim 1, wherein the compound of Formula (I) is selected from

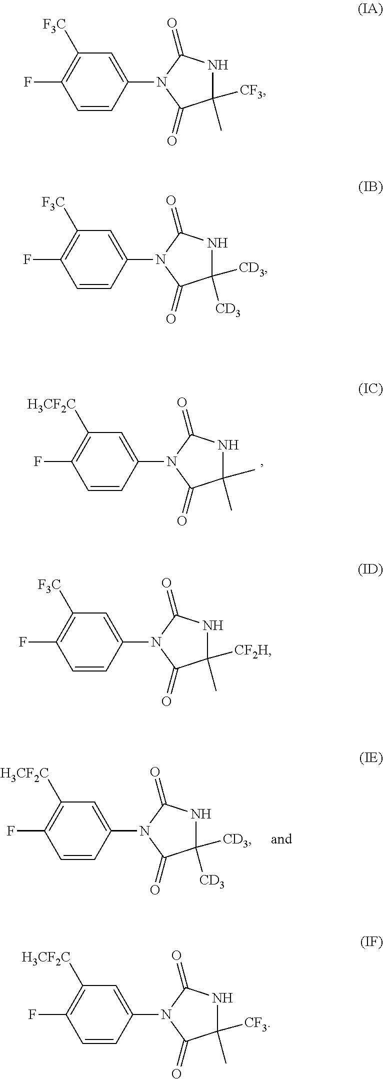

11. The compound or pharmaceutically acceptable salt of claim 10, wherein the compound of Formula (I) is Formula (IE) or Formula (IF).

12. The compound or pharmaceutically acceptable salt of claim 11, wherein the compound of Formula (I) is Formula (IE).

13. A pharmaceutical composition comprising a compound or pharmaceutically acceptable salt according to claim 1 and a pharmaceutically acceptable carrier or excipient.

14. The pharmaceutical composition of claim 13, wherein the compound of Formula (I) is selected from a compound of Formula (IA), Formula (IB), Formula (IC), Formula (ID), Formula (IE), and Formula (IF).

15. The pharmaceutical composition of claim 14, wherein the compound of Formula (I) is a compound of Formula (IE) or Formula (IF).

16. The pharmaceutical composition of claim 15, wherein the compound of Formula (I) is Formula (IE).

17. The pharmaceutical composition of claim 13, further comprising at least one vaccine antigen.

18. A method of treating, inhibiting, and/or preventing a parasitic disease in a subject in need thereof, wherein the method comprises administering to said subject an effective amount of a compound or pharmaceutically acceptable salt according to claim 1.

19. The method of claim 18, wherein the compound of Formula (I) is selected from a compound of Formula (IA), Formula (IB), Formula (IC), Formula (ID), Formula (IE), and Formula (IF).

20. The method of claim 19, wherein the compound of Formula (I) is a compound of Formula (IE) or Formula (IF).

21. The method of claim 20, wherein the compound of Formula (I) is Formula (IE).

22. The method of claim 18, wherein the parasitic disease is Schistosomiasis.

23. The method of claim 18, wherein the compound of Formula (I) is administered orally.

24. The method of claim 18, wherein the compound of Formula (I) is administered with at least one vaccine antigen.

25. The composition of claim 13 for use in treating, inhibiting, and/or preventing a parasitic disease in a subject in need thereof.

26. The composition for use of claim 25, wherein the compound of Formula (I) is selected from a compound of Formula (IA), Formula (IB), Formula (IC), Formula (ID), Formula (IE), and Formula (IF).

27. The composition for use of claim 26, wherein the compound of Formula (I) is a compound of Formula (IE) or Formula (IF).

28. The composition for use of claim 27, wherein the compound of Formula (I) is a compound of Formula (IE).

29. The composition for use of claim 13, wherein the disease is Schistosomiasis.

30. The composition of claim 17 for use in treating, inhibiting, and/or preventing a parasitic disease in a subject in need thereof.

31. The composition for use of claim 30, wherein the compound of Formula (I) is selected from a compound of Formula (IA), Formula (IB), Formula (IC), Formula (ID), Formula (IE), and Formula (IF).

32. The composition for use of claim 31, wherein the compound of Formula (I) is a compound of Formula (IE) or Formula (IF).

33. The composition for use of claim 32, wherein the compound of Formula (I) is a compound of Formula (IE).

34. The composition for use of claim 30, wherein the disease is Schistosomiasis.

35. Use of the composition of claim 13 for treating, inhibiting, and/or preventing a parasitic disease in a subject in need thereof.

36. The use of claim 35, wherein the compound of Formula (I) is selected from a compound of Formula (IA), Formula (IB), Formula (IC), Formula (ID), Formula (IE), and Formula (IF).

37. The use of claim 36, wherein the compound of Formula (I) is a compound of Formula (IE) or Formula (IF).

38. The use of claim 37, wherein the compound of Formula (I) is a compound of Formula (IE).

39. The use of claim 35, wherein the parasitic disease is Schistosomiasis.

40. Use of the composition of claim 17 for treating, inhibiting, and/or preventing a parasitic disease in a subject in need thereof.

41. The use of claim 40, wherein the compound of Formula (I) is selected from a compound of Formula (IA), Formula (IB), Formula (IC), Formula (ID), Formula (IE), and Formula (IF).

42. The use of claim 41, wherein the compound of Formula (I) is a compound of Formula (IE) or Formula (IF).

43. The use of claim 42, wherein the compound of Formula (I) is a compound of Formula (IE).

44. The use of claim 40, wherein the parasitic disease is Schistosomiasis.
